(19) United States
(12) Patent Application Publication
(10) Pub. No.: US 2012/0129939 A1
(43) Pub. Date: May 24, 2012

(54) POLYAMINE INHIBITORS FOR THE TREATMENT AND PREVENTION OF PARKINSON'S DISEASE

(76) Inventors: Scott A. Small, Millterton, NY (US); Nicole Lewandowski, New York, NY (US); Donald W. Landry, New York, NY (US); Shi-Xiang Deng, White Plains, NY (US)

(21) Appl. No.: 13/203,649

(22) PCT Filed: Mar. 1, 2010
(86) PCT No.: PCT/US10/00613

§ 371 Date: Feb. 9, 2012

Related U.S. Application Data
(60) Provisional application No. 61/208,879, filed on Feb. 26, 2009.

(51) Int. Cl. A61K 31/197; A61P 25/16; C07D 243/04; C07D 241/08; A61K 31/132; C07C 229/26

(52) U.S. Cl. 514/564; 514/674; 560/169; 562/561; 540/492; 544/385

(57) ABSTRACT

Disclosed herein are methods for treating a disease involving α-synucleic aggregation using (1) a compound which reduces the amount of polyamines in an amount effective to reduce α-synucleic aggregation; (2) a compound which inhibits polyamine synthesis in an amount effective to reduce α-synucleic aggregation; or (3) a compound which inhibits α-synucleic aggregation in an amount effective to reduce α-synucleic aggregation. Also disclosed are methods for reducing the amount of α-synucleic aggregation in a brain cell using (1) a compound which reduces the amount of polyamines in an amount effective to reduce α-synucleic aggregation; (2) a compound which inhibits polyamine synthesis in an amount effective to reduce α-synucleic aggregation; or (3) a compound which inhibits α-synucleic aggregation in an amount effective to reduce α-synucleic aggregation. Disclosed herein are also compounds which can be used in the above described methods.

This application claims priority of U.S. Provisional Application No. U.S. Provisional Application No. 61/208,879, filed Feb. 26, 2009, the content of which is hereby incorporated by reference.

Throughout this application various publications and published patents are referenced by author name and year or by patent application publication number or patent number. A complete list of these references appear before the claims. The disclosures of these publications in their entireties are hereby incorporated by reference into this application in order to more fully describe the state of the art to which this invention pertains.

The invention disclosed herein was made with government support from the National Institutes of Health (NIH) grants NS36630. AG18440, AG02207, and UL1 RR0241456. Accordingly, the U.S. Government has certain rights in this invention.

### BACKGROUND OF INVENTION

Parkinson's disease (PD) is the second most common neurodegenerative disease. Although many of the pathogenic molecules underlying the rare autosomal dominant forms of PD have been identified (Hardy et al. 2006), the full complement of pathogenic pathways involved in the common “sporadic” form of PD remains unknown (Litvan et al. 2007). In principle, gene expression profiling techniques like microarray are well suited to identify molecular pathways contributing to the pathogenesis of complex diseases (Coppola, G. and Geschwind, D. H 2006). In practice, however, microarray applied to diseases of the brain present a number of analytic challenges (Lewandowski et al. 2005). By identifying regions within the same brain structure that are differentially targeted by and resistant to a disease, imaging-guided microarray is an approach designed to address these limitations (Lewandowski et al. 2005). Specifically, guided by the spatial information generated from high-resolution functional imaging, a 2×2 factorial analysis-of-variance can be designed, including both within and between group factors, and this “double subtraction” model is effective in improving signal-to-noise in a microarray experiment (Kerr et al. 2000). For example, this approach has been successfully used in identifying retromer sorting as a pathogenic mechani in late onset Alzheimer's disease (Small, S. et al. 2005).

### Parkinson's Disease Background

Parkinson's disease (PD) is one of the most severe and widespread age-related neurodegenerative disorders, affecting almost 1% of the population aged over 65 years. PD is characterized clinically by resting tremor, rigidity, postural instability, bradykinesia, impaired balance, and coordination (Lew, M. 2007). These symptoms have an insidious, progressive, and characteristically asymmetric onset. Clinical diagnosis of idiopathic Parkinson's disease rests on these features, with definitive diagnosis currently being possible only on the basis of postmortem pathologic findings.

Although classically, motor abnormalities are considered the hallmark of the disease, there is also evidence of nonmotor impairments in PD (Witjas et al., 2007; Przutnek, H. et al., 2004; and Pfeiffer, R. F. 2003). A large proportion of patients have varying degrees of neuropsychiatric (depression, apathy, anxiety, abulia, psychosis), cognitive disturbances (dementia, slowing of thought processes, difficulty with abstract thought), insomnia, dystonia, and muscle aches (Fahn, S. R. 1987). Patients also display gastrointestinal symptoms, such as; constipation, nausea, salivary excess, dysphagia, gastroparesis, and intestinal dysmotility (Pfeiffer, R. F. 2003). Some other nonmotor impairments involve dysfunction within the autonomic nervous system. These are symptoms that appear early on and/or develop as the disease progresses: Urinary urgency and frequency, as well as incontinence, orthostatic hypotension, seborrheic dermatitis, oily skin, hyperhidrosis, sialorrhea, and sexual dysfunction (Lew, M. 2007). The occurrence of some of these symptoms may, in part, be due to the side effects of pharmacological treatment. Although, it should be noted that many of these symptoms present before the onset of the classic motor impairments, and thus are present before diagnosis and drug intervention. For this reason, it is probable to hypothesize that if intervention can occur at this early symptomatic stage before diagnosis, we may be able to diminish the severity of future symptoms of PD, or better yet, prevent its progression all together. In order to do this we must understand why these early symptoms occur and the corresponding pathology that is involved.

### Pathology of Parkinson's Disease

The hallmark of PD is preferential degeneration of the dopaminergic neurons, which can lead to hypodopamine transmission in the substantia nigra pars compacta (SNpc). Nigral cell death is accompanied by the accumulation of a wide range of poorly degraded proteins and the formation of proteinaceous inclusions (Lewy neurites and bodies) in dopaminergic neurons (Formo, L. 1996).

Several different genetic factors encoding, alpha-synuclein, parkin, DJ-1, PINK-1, and LRRK2, are known to account for a small number of familial cases of PD, but the overwhelming majority of patients lack a positive family history and have a sporadic form of the disease. In these cases, there is evidence supporting a role for oxidative stress, excitotoxicity, and mitochondrial dysfunction in the cascade of events leading to dopaminergic neuronal (DN) death (Mytilineou, C. et al. 2004). However, these defects are not thought to be the primary cause of neurodegeneration since in experimental models they induce dopaminergic neuronal death without Lewy neurite (LN) or Lewy body (LB) formation (Gerlach, M. and Rieferer, P. 1996).

α-Synuclein has been the protein of focus in PD research. This is because α-synuclein has an increased propensity to aggregate due to its hydrophobic non-amyloid-β component domain, and the presence of fibrillar α-synuclein as a major structural component of LN/LB in PD suggests a role of aggregated α-synuclein in disease pathogenesis (Spillantini, M. G. et al. 1998). Mechanisms by which abnormal processing and accumulation of α-synuclein disrupt basic cellular functions leading to dopaminergic neurodegeneration are intensely studied. One of the earliest defects following α-synuclein accumulation in vivo is blockade of endoplasmic reticulum to golgi vesicular trafficking causing ER stress (Cooper, A. A. et al. 2006). Furthermore, transgenic mice expressing human A53T α-synuclein (a common PD mutation) develop mitochondrial pathology (Martin, L. J. 2006) providing a crucial role of α-synuclein in modulating mitochondrial function in neurodegeneration. This may be due to the fact that α-synuclein is a modulator of oxidative damage, since mice lacking α-synuclein are resistant to mitochondrial toxins (Klivenyi, P. et al. 2006), while nigral dopaminergic neurons are vulnerable to degeneration and mitochondrial dysfunction following parkinsonian neurotoxin MPTP (1-methyl-4-phenyl-1,2,3,6-tetrahydropyridine) treatment in human α-synuclein transgenic mice (Nieto, M. et al. 2006). Furthermore, mutant α-synuclein (A53T and A30P) overexpression increases cytosolic catecholamine concentrations leading to disruption of vesicular pH and normal functioning, and facilitate toxicity of oxidized catechol metabolites implicating selective degeneration in PD (Mosharov, E. V. et al. 2006). Biochemical abnormalities in α-synuclein have also been shown to, activate stress-signaling protein kinases (Klegeris, A. et al. 2006), affect age-related decrease in neurogenesis (Winner, B. et al. 2006), impair microtubule-dependent trafficking (Lee, H. J. et al, 2006), reduce intercellular communications at gap junctions (Sung, J. Y. et al. 2007), and inhibit histone acetylation in the nucleus to promote toxicity (Kontopoulos, E. et al. 2006). These pathophysiological aspects are detrimental to normal functioning of dopaminergic neurons and provide implications for disease pathogenesis in α-synuclein-induced PD.

With these findings and the high propensity of α-synuclein inclusions found within the SNpc, PD research lead to multiple studies within the SNpc. These studies showed that in PD stage development, α-synuclein inclusions are not seen in SNpc until later on (stage 3, based on Braak & Braak pathological staging of PD) in the disease (Braak, H. et al. 2003). Early intervention is the key to diminishing progression. Studies have shown that the first sight of PD pathology is seen within the dorsal motor nucleus of the vagus (Braak, H. et al. 2003).

### Dorsal Motor Nucleus of the Vagus Nerve (X)

Within the medulla oblongata lies the dorsal motor nucleus of the vagus nerve (X), DMNV. During stage 1 of PD pathology distinctive inclusion bodies that present in the form of spindle-like or thread-like branching within cellular processes, known as Lewy neurites (LN), are present within the DMNV (Braak, H. et al. 2003). These LN are sometimes referred to as LB precursors. The difference is LN, as described, are thread-like within the cellular processes while LB are granular aggregations and spherical pale bodies in the somata. In general LN form before LB, thus when discussing early stages of pathology, the α-synuclein inclusions that are seen, are that of LN.

Even though the DMNV is the first observed sight of PD pathology, it remains to be investigated for during stage 1 patients are unsymptomatic, that is there is no motor dysfunction seen, and therefore PD is not even a clinical suspicion at this point (Braak et al. 2004). Although, as was mentioned prior, the autonomic nervous system is affected in PD early on, and therefore DMNV pathology may be clinically relevant. The DMNV has preganglion parasympathetic fibers that innervate numerous organs (Dorsal Motor Nucleus of the Vagus 2008) and dysfunction in these organs can lead to the nonmotor autonomic symptoms that are seen in PD. This along with its early histological pathology makes the DMNV a desired area of investigation.

### Parkinson's Disease and the Polyamine Pathway

Although subregions of the basal ganglia have been clearly implicated in PD, in the current study we decided to focus on subregions within the medulla for a number of reasons. First, although still controversial, postmortem studies that measure α-synuclein inclusions, a histological hallmark of PD, suggest that the dorsomedial medulla might be affected relatively early in the course of the disease (Braak, H. et al. 2003). Second, and more importantly, α-synuclein inclusions in the medulla are associated with relatively less gliosis and cell death, factors that confound the interpretation of gene-expression studies. At the same time, however, the lack of cell death has raised the question whether α-synuclein inclusions in regions within the medulla are functionally benign, although early non-motor PD symptoms can localize to medullary nuclei, such as the dorsal motor nucleus of the vagus (DMNV) (Simunit, T. & Sethi, K. 2008; Pfeiffer, R. F. 2003).

### SUMMARY OF THE INVENTION

The present invention provides a method for treating a subject afflicted with a disease involving α-synucleic aggregation which comprises administering to the subject a compound which reduces the amount of polyamines in the subject in an amount effective to reduce α-synucleic aggregation so as to thereby treat the subject. In one embodiment of the invention the disease is Parkinson's Disease.

This invention also provides a method for treating a subject afflicted a disease involving α-synucleic aggregation which comprises administering to the subject a compound which inhibits polyamine synthesis in the subject in an amount effective to reduce α-synucleic aggregation so as to thereby treat the subject.

This invention also provides a method for a subject afflicted with a disease involving α-synucleic aggregation which comprises administering to the subject a compound which inhibits α-synucleic aggregation in the subject in an amount effective to reduce α-synucleic aggregation so as to thereby treat the subject.

This invention also provides a method for reducing the amount of α-synucleic aggregation in a brain cell which comprises treating the brain cell with a compound that reduces the amount of polyamines in the brain cell so as to thereby reduce the amount of α-synucleic aggregation in the brain cell.

This invention also provides a method for reducing the amount of α-synucleic aggregation in a brain cell which comprises treating the brain cell with a compound that inhibits polyamine synthesis so as to thereby reduce the amount of α-synucleic aggregation in the brain cell.

This invention also provides a method for reducing α-synucleic aggregation in a brain cell which comprises treating the brain cell with a compound that inhibits α-synucleic aggregation so as to thereby reduce α-synucleic aggregation in the brain cell.

This invention also provides a compound having the structure:

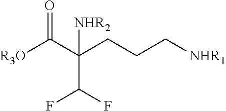

wherein R₁ is H,

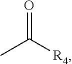

or
wherein R₄ is OH, OR₅, SR₅ or NHR₅;
wherein R₅ is a substituted or unsubstituted alkyl, or a C₂-C₅ alkyl:
wherein R₂ is H,

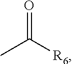

wherein R₆ is OH, OR₇, SR₇ or NHR₇;
wherein R₇ is a substituted or unsubstituted alkyl, or a C₁-C₅ alkyl;
wherein R₃ is H, a substituted or unsubstituted alkyl, or a C₁-C₅ alky;
or a salt or a pharmaceutically acceptable thereof.

This invention also provides a compound having the structure:

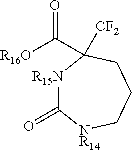

wherein
R₁₄ is H, substituted or unsubstituted alkyl, C₁-C₅ alkyl;
R₁₅ is H, substituted or unsubstituted alkyl, C₁-C₅ alkyl; and
R₁₆ is H, substituted or unsubstituted alkyl, C₁-C₅ alkyl;
or a salt or a pharmaceutically acceptable salt thereof.

This invention also provides a compound having the structure:

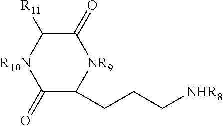

wherein

### R₈ is H or

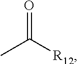

wherein R₁₂ is OH, OR₁₃, SR₁₃, or NHR₁₃
wherein R₁₃ is H, substituted or unsubstituted alkyl or C₁-C₅ alkyl;
R₉ is H or substituted or unsubstituted alkyl or C₁-C₅ alkyl;
R₁₀ is H or substituted or unsubstituted alkyl or C₁-C₅ alkyl; and
R₁₁ is H or substituted or unsubstituted alkyl;
or a salt or a pharmaceutically acceptable salt thereof.

### DESCRIPTION OF THE FIGURES

FIG. 1A-1C. Functional imaging identifies brainstem regions targeted by and resistant to PD. FIG. 1A. Anatomical criteria for slice selection and cerebral blood volume maps of the medulla identify selective dysfunction in the dorsal motor nucleus of the vagus (DMNV). (A) Anatomical criteria used to identify an MRI slice of the medulla that contains the DMNV. As shown by the sagittal scout image (left pane), MRI slices were acquired from anterior to posterior (three consecutive MRI slices are shown together with their accompanying histological slices) perpendicular to the long-axis of the brainstem (black line in scout image). Using strict anatomical criteria (see methods), a single slice (red line in scout image and red-boxed middle images) was identified in each individual subject, containing the DMNV (red circle in the middle boxed histological slice). FIG. 1B Contrast-enhanced signal change in the sigmoid sinus (demarcated by green) was used to generate percent CBV maps of the medulla (% CBV) (upper image). Results from the unbiased analysis confirmed that the site of PD-related dysfunction is in the DMNV (lower image). For anatomical reference, the z-score map, comparing CBV maps of PD cases versus controls, is shown on the right with the associated histological image shown on the left. The z-score map is color coded (color bar on the right), such that warmer colors indicated higher CBV. FIG. 1C Representation of region-of-intrest measurements along the DMNV (upper image). The gray line demarcates the midline of the medulla, three ROIs were created on the left and right side. The boxes represent the three measurments, the box furthest from the midline is the lateral measure, the middle box represents the central measure, and the box closest to the midline represents the medial measure. Results from the hypothesis driven analysis identified PD-related dysfunction in the central dorsal medulla (lower graph). The y-axis=% CBV for each ROI (lateral, central, and medial) averaged for PD cases and controls, of both the left and right side of the medulla.

FIG. 2A-2C. Expression profiles of Spermidine/spermine N1-acetyltransferease and other molecules are differentially affected in Parkinson's disease. FIG. 2A Ten molecules whose expression levels via microarray analysis were differentially affected in PD cases versus controls, comparing the dorsal motor nucleus of the vagus (DMNV) to the inferior olivary nucleus (ION). Note, that for all 10 transcripts the expression level was downregulated in PD vs. controls, p-values were calculated by a repeated-measures 2×2 factorial ANOVA. FIG. 2B Mean expression levels are shown on the graph for Spermidine/spermine N1-acetyltransferease for mRNA where n=22 (6 DMNV and 6 ION for PD and 5 for each region in controls). Expression within the DMNV was significantly down in PD compared to controls (p=0.002) (left graph). In a new set of brains, protein expression via Western blot, where n=20 (5 DMNV and 5 ION for each group, control and PD), showed a significant decrease of SAT1 expression (p=0.045) in the DMNV of PD samples compared to controls, but not in the ION, as seen in the blot image of representative samples and the right graph. FIG. 2C Immunohistochemistry for SAT1 protein in the DMNV (left panels) and the ION (right panels) showed positive expression within the neuronal cell bodies of both regions, at 40× (left panels) and 400× (right panels) respectively.

FIG. 3. Yeast studies validate the importance of polyamines in PD FIG. 3A Yeast cells integrated with empty vector (Vec), wildtype α-synuclein (Syn-WT), or mutant α-synuclein (Syn-A53T) were grown in YPGaI medium supplemented with spermine at concentrations of 0 mM (upper graph), 0.2 mM (middle graph), and 0.4 mM (lower graph). OD600 was monitored at one hour intervals using the Bioscreen system. The growth curves shown above were representative of three independent experiments. FIG. 3B In an unbiased genome-wide yeast screen, Tpo4 was shown to enhance toxicity in cells expressing α-synuclein at a ˜40% higher level (IntTox); in comparison to a known toxicity enhancer (Gyp8) and suppressor (Ypt1). FIG. 3C Tpo4 caused α-synuclein to form intracellular foci more rapidly than it otherwise would in the IntTox strain.

FIG. 4A-4C. Mice studies validate that SAT1 activity modifies PD pathology Transgenic mice that express wildtype human α-synuclein and controls were treated by intracranial infusion via osmotic pumps with PBS, DENSPM, or Berenil for six weeks. Results for immunoreactivity within the basal ganglia are shown. FIG. 4A Mean α-synuclein cell pixel intensity (cell mean) in the caudo-putamen was increased with Berenil (* where p=0.009) and decreased with DENSPM (* where p=0.043) in comparison to transgenic α-synuclein mice treated with PBS (syntgPBS). FIG. 4B Tyrosine hydroxylase (TH) fibers were decreased with Berenil treatment (* where p=0.033), and increased with DENSPM (* where p=0.022), compared to syntgPBS, as shown by the cell mean (TH corrected optical density in the caudo-putamen). FIG. 4C Berenil caused a decrease in MAP2 (* where p=0.013), compared to syntgPBS, while DENSPM rescued the transgenic α-synuclein effects. % Cell mean represents the % of the neuropil covered by MAP2 immunoreactive dendrites in the caudo-putamen.

FIG. 5A-5C. Genetic studies in human patients identifies a novel PD-associated mutation in SAT1 FIG. 5A Schematic of the SAT1 gene and the location of the deletion within the 3′UTR of SAT1. FIG. 5B Sequence chromatograms showing a PD patient heterozygous for the c.786_{—}788delTGT variant and FIG. 5C a wildtype subject

FIG. 6. Mice Studies Validate that SAT1 activity modified PD pathology Trangenic mice that express wildtype human α-synuclein and control were treated by intracranial infusion via osmotic pumps with PBS, DENSPM, or Berenil for six (6) weeks. Results for immunoreactivity within the substantia nigra are shown. Mean α-synuclein cell pixel intensity (cell mean) was increased with berenil (* where p=0.001) and a trending decrease seen with DENSPM in comparison to transgenic α-synuclein mice treated with PRB (syntgPBS).

FIG. 7. Standard Curve Graph 1 This figure shows the standard curve for putrescine, spermine and spermidine prepared for HPLC experiments.

FIG. 8. Standard Curve Graph 2 This figure shows the standard curve for putrescine, spermine and spermidine prepared for HPLC experiments.

### DETAILED DESCRIPTION OF THE INVENTION

The present invention provides a method for treating a subject afflicted with a disease involving α-synucleic aggregation which comprises administering to the subject a compound which reduces the amount of polyamines in the subject in an amount effective to reduce α-synucleic aggregation so as to thereby treat the subject. In one embodiment of the invention the disease is Parkinson's Disease.

In a further embodiment of the method the compound reduces the amount of polyamines in the subject by inhibiting ornithine decarboxylase. In one embodiment the compound is α-difluoromethylornithine (DEMO), or a salt or pharmaceutically acceptable salt thereof. In another embodiment the compound is an analog of α-difluoromethylornithine (DEMO). In a further embodiment the compound is DEMO analog 1, DEMO analog 2, DEMO analog 3, or DEMO analog 4, each of which are described herein.

In another embodiment of the method, the compound reduces the amount of polyamines in the subject by inducing SSAT1 synthesis in the subject. In one embodiment, the compound is an analog of a polyamine. In a further embodiment the compound is N1, N11-diethynorspermine (DENSPM).

In one embodiment of the method the administration is intrathecal administration.

This invention also provides a method for treating a subject afflicted a disease involving α-synucleic aggregation which comprises administering to the subject a compound which inhibits polyamine synthesis in the subject in an amount effective to reduce α-synucleic aggregation so as to thereby treat the subject.

In a further embodiment of the method the compound reduces the amount of polyamines in the subject by inhibiting ornithine decarboxylase. In one embodiment the compound is α-difluoromethylornithine (DFMO), or a salt or pharmaceutically acceptable salt thereof. In another embodiment the compound is an analog of α-difluoromethylornithine (DFMO). In a further embodiment the compound is DFMO analog 1, DFMO analog 2, DFMO analog 3, or DFMO analog 4, each of which are described herein.

In one embodiment of the method, the administration is intrathecal administration.

This invention also provides a method for a subject afflicted with a disease involving α-synucleic aggregation which comprises administering to the subject a compound which inhibits α-synucleic aggregation in the subject in an amount effective to reduce α-synucleic aggregation so as to thereby treat the subject.

In a further embodiment of the method the compound reduces the amount of polyamines in the subject by inhibiting ornithine decarboxylase. In one embodiment the compound is α-difluoromethylornithine (DFMO), or a salt or pharmaceutically acceptable salt thereof. In another embodiment the compound is an analog of α-difluoromethylornithine (DFMO). In a further embodiment the compound is DFMO analog 1, DFMO analog 2, DFMO analog 3, or DFMO analog 4, each of which are described herein.

In another embodiment of the method, the compound reduces the amount of polyamines in the subject by inducing SSAT1 synthesis in the subject. In one embodiment, the compound is an analog of a polyamine. In a further embodiment the compound is N1, N11-diethynorspermine (DENSPM).

In one embodiment of the method, the administration is intrathecal administration.

This invention also provides a method for reducing the amount of α-synucleic aggregation in a brain cell which comprises treating the brain cell with a compound that reduces the amount of polyamines in the brain cell so as to thereby reduce the amount of α-synucleic aggregation in the brain cell.

In one embodiment of the method the brain cell is a dorsal motor nucleus of the vagus (DOMV) brain cell. In another embodiment the brain cell is an inferior olivary nucleus brain cell.

In a further embodiment of the method the compound reduces the amount of polyamines in the subject by inhibiting ornithine decarboxylase. In one embodiment the compound is α-ditluoromethylornithine (DEMO), or a salt or pharmaceutically acceptable salt thereof. In another embodiment the compound is an analog of α-difluoromethylornithine (DEMO). In a further embodiment the compound is DEMO analog 1, DEMO analog 2, DEMO analog 3, or DEMO analog 4, each of which are described herein.

In another embodiment of the method, the compound reduces the amount of polyamines in the subject by inducing SSAT1 synthesis in the subject. In one embodiment, the compound is an analog of a polyamine. In a further embodiment the compound is N1, N11-diethynorspermine (DENSPM).

This invention also provides a method for reducing the amount of α-synucleic aggregation in a brain cell which comprises treating the brain cell with a compound that inhibits polyamine synthesis so as to thereby reduce the amount of α-synucleic aggregation in the brain cell.

In one embodiment of the method the brain cell is a dorsal motor nucleus of the vagus (DOMV) brain cell. In another embodiment the brain cell is an inferior olivary nucleus brain cell.

In a further embodiment of the method the compound reduces the amount of polyamines in the subject by inhibiting ornithine decarboxylase. In one embodiment the compound is α-difluoromethylornithine (DEMO), or a salt or pharmaceutically acceptable salt thereof.

In another embodiment the compound is an analog of α-difluoromethylornithine (DFMO). In a further embodiment the compound is DEMO analog 1, DFMO analog 2, DFMO analog 3, or DFMO analog 4, each of which are described herein.

In another embodiment of the method, the compound reduces the amount of polyamines in the subject by inducing SSAT1 synthesis in the subject. In one embodiment, the compound is an analog of a polyamine. In a further embodiment the compound is N1, N11-diethynorspermine (DENSPM).

This invention also provides a method for reducing α-synucleic aggregation in a brain cell which comprises treating the brain cell with a compound that inhibits α-synucleic aggregation so as to thereby reduce α-synucleic aggregation in the brain cell.

In one embodiment of the method the brain cell is a dorsal motor nucleus of the vagus (DOMV) brain cell. In another embodiment the brain cell is an inferior olivary nucleus brain cell.

In a further embodiment of the method the compound reduces the amount of polyamines in the subject by inhibiting ornithine decarboxylase. In one embodiment the compound is α-difluoromethylornithine (DFMO), or a salt or pharmaceutically acceptable salt thereof. In another embodiment the compound is an analog of α-difluoromethylornithine (DFMO). In a further embodiment the compound is DFMO analog 1, DFMO analog 2, DFMO analog 3, or DFMO analog 4, each of which are described herein.

In another embodiment of the method, the compound reduces the amount of polyamines in the subject by inducing SSAT1 synthesis in the subject. In one embodiment, the compound is an analog of a polyamine. In a further embodiment the compound is N1, N11-diethynorspermine (DENSPM).

This invention also provides a compound having the structure:

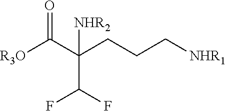

wherein R₁ is H, or

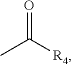

wherein R₄ is OH, OR₅, SR₅ or NHR₅;
wherein R₅ is a substituted or unsubstituted alkyl, or a C₁-C₅ alkyl;
wherein R₂ is H,

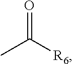

wherein R₆ is OH, OR₇, SR₇ or NHR₇;
wherein R₇ is a substituted or unsubstituted alkyl, or a C₁-C₅ alkyl;
wherein R₃ is H, a substituted or unsubstituted alkyl, or a C₁-C₅ alky;
or a salt or a pharmaceutically acceptable thereof.

In one embodiment of the compound

### R₁ is H or

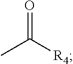

### R₂ is H or

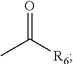

and R₃ is alkyl; or a salt or a pharmaceutically acceptable salt thereof.

In another embodiment the compound is DFMO Analog 1 having the structure:

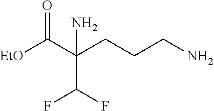

or a salt or a pharmaceutically acceptable salt thereof.

In another embodiment of the compound,

### R₁ is

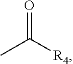

wherein R₄ is OH, OR₅, SR₅ or NHR₅,
wherein R₅ is a substituted or unsubstituted alkyl, or a C₁-C₅ alkyl;
wherein R₇ is a substituted or unsubstituted alkyl, or a C₁-C₅ alkyl;

### R₂ is

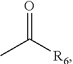

wherein R₆ is OH, OR₇, SR₇ or NHR₇,
wherein R₇ is a substituted or unsubstituted alkyl, or a C₁-C₅ alkyl;
wherein R₃ is alkyl;
or a salt or a pharmaceutically acceptable salt thereof.

In another embodiment the compound is DFMO Analog 2 having the structure:

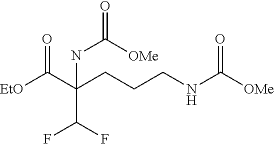

or a salt or a pharmaceutically acceptable salt thereof.

This invention also provides a compound having the structure:

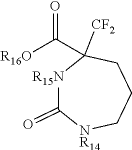

wherein
R₁₄ is H, substituted or unsubstituted alkyl, C₁-C₅ alkyl;
R₁₅ is H, substituted or unsubstituted alkyl, C₁-C₅ alkyl; and
R₁₆ is H, substituted or unsubstituted alkyl, C₁-C₅ alkyl;
or a salt or a pharmaceutically acceptable salt thereof.

In one embodiment of the compound

### R₁₅ is H;

### R₁₄ is H; and

R₁₆ is alkyl;
or a salt or a pharmaceutically acceptable salt thereof.

In another embodiment the compound is DEMO Analog 3 having the structure:

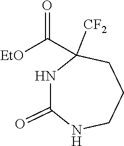

or a salt or a pharmaceutically acceptable salt thereof.

This invention also provides a compound having the structure:

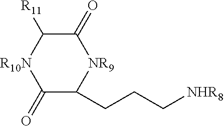

wherein

### R₈ is H or

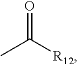

wherein R₁₂ is OH, OR₁₃, SR₁₃, or NHR₁₃
wherein R₁₃ is H, substituted or unsubstituted alkyl or C₁-C₅ alkyl;
R₉ is H or substituted or unsubstituted alkyl or C₁-C₅ alkyl;
R₁₀ is H or substituted or unsubstituted alkyl or C₁-C₅ alkyl; and
R₁₁ is H or substituted or unsubstituted alkyl;
or a salt or a pharmaceutically acceptable salt thereof.

In one embodiment of the compound

### R₈ is H or

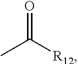

wherein R₁₂ is OH, OR₁₃, SR₁₃, or NHR₁₃
wherein R₁₃ is H, substituted or unsubstituted alkyl or C₁-C₅ alkyl;

### R₉ is H;

### R₁₀ is H; and

R₁₁ is H or substituted or unsubstituted alkyl;
or a salt or a pharmaceutically acceptable salt thereof.

In another embodiment the compound is DFMO Analog 4 having the structure:

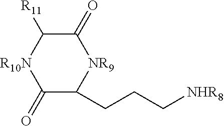

wherein R₈ is H or

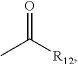

wherein R₁₁ is H or substituted or unsubstituted alkyl;
or a salt or a pharmaceutically acceptable salt thereof.

### DEFINITIONS

As used in this application, except as otherwise expressly provided herein, each of the following terms shall have the meaning set forth below.

As used herein, “α-synucleic aggregation” shall mean when normally soluble α-synucleic proteins or protein fragments aggregate to form insoluble fibers often characterized by Lewy bodies.

As used herein, “diseases involving α-synucleic aggregation” include but are not limited to diseases characterized by the presence of Lewy bodies. These may include but are not limited to Parkinson's disease, dementia with Lewy bodies, diffuse Lewy body disease, amyotrophic lateral disease, multiple system atrophy and Alzheimer's disease.

As used herein, “polyamines” shall mean organic compounds having two or more primary amino groups which are synthesized in the cells in the polyamine pathway. Polyamines may include, but are not limited to, putrescine, cadaverine, spermidine, and spermine.

Polyamine analogs are known in the art and described, for exampled in, Wallace, H. M. and Kiiranen, K. “Polyamine analogues—an update” Amino Acids (2007) 33:261-265, the contents of which are hereby incorporated by reference, and include for example, but are not limited to, AOE-PU, N-[2-aminooxyethyl]-1,4-diaminobutane; APA, 1-aminooxy-3-aminopropane; AP-APA, 1-aminooxy-3-N-[3-aminoproplyl]-aminopropane; BCNU, 1-3-bis(2-chloroethyl)-1-nitrosurea; BEHSpm/DEHSpm/BE-4-4-4, N¹,N¹⁴-bis(ethyl)-homospermine; BENSpm/DENSpm/BE-3-3-3, N¹,N¹¹-bis(ethyl)-norspermine; BES, N1,N8-bis(ethyl)spermidine; BESpm/DESpm/BE-3-4-3, N¹,N¹²-bis(ethyl)spermine; CHENSpm, N¹-ethyl-N¹¹-(ccycloheptyl)methyl)-4,8-diazaundecane; IPENSpm, N¹-ethylN¹¹-((isopropyl)-methyl)-4-8-dizaundecane; MGBG, methylglyoxal bis(guanylhydrazone). Other polyamine analogs are 3,8,13,18-tetraaza-10,11-[(E)-1,2-cyclopropyl]eicosane Tetrahydrocholride, which is also known as CGC-11093 (Ignatenko et al. 2006) or ([¹N,¹²N]Bis(Ethyl)-cis-6,7-Dehydrospermine. Other polyamine analogs include CGC011157, CGC-11158, CGC-11144, and CGC-11047, the structures of which are described and provided in Yarlett et al. 2007, the contents of which are hereby incorporated by reference. Still further polyamine analogs and methods of making polyamine analogs are described in U.S. Pat. No. 7,312,244; U.S. Pat. No. 7,279,502; U.S. Pat. No. 7,186,825, U.S. Pat. No. 6,982,351, U.S. Pat. No. 7,453,011, U.S. Pat. No. 6,794,545, U.S. Pat. No. 6,649,587; U.S. Pat. No. 7,235,695; U.S. Pat. No. 6,872,852; U.S. Patent Application Publication No. 2004/0006049; U.S. Patent Application Publication No. 2003/0130356, U.S. Patent Application Publication No. 2009/0275664; U.S. Patent Application Publication No. 2009/0134456; U.S. Patent Application Publication No. 2007/0232677; U.S. Patent Application Publication No. Application Publication No. 2002/0143068; and U.S. Patent Application Publication No. 2002/0045780, the contents of all of which are hereby incorporated by reference. In one embodiment of the methods described herein, the compound is any one of the above-described polyamine analogs.

As used herein, “polyamine pathway” shall mean the molecular pathway involved in the synthesis and metabolism of polyamines including putrescine, cadaverine, spermidine and spermine.

As used herein, “brain cell” shall mean a cell found in or derived from a cell originating in the brain of an animal including a human. Examples of brain cells include cells found in or derived from the dorsal motor nucleus of the vagus (DMNV) and the inferior olivary nucleus (ION).

As used herein, “administering” an agent can be effected or performed using any of the various methods and delivery systems known to those skilled in the art. The administering can be performed, for example, intravenously, intraperitoneally, via cerebrospinal fluid, orally, nasally, via implant, transmucosally, transdermally, intramuscularly, and subcutaneously.

The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “subject” shall mean any animal, such as a human, non-human primate, mouse, rat, guinea pig or rabbit.

As used herein, “treating” shall mean slowing, stopping or reversing the progression of a disorder or disease.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, and butenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, the term “cycloalkyl” refers to a monocyclic, bicyclic, or tricyclic ring system, which may be saturated or partially saturated, i.e. possesses one or more double bonds. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclopentenyl, cyclohexyl, cyclohexenyl, cycloheptyl and cyclooctyl. Bicyclic fused ring systems are exemplified by a cycloalkyl ring fused to another cycloalkyl ring. Examples of bicyclic fused ring systems include, but are not limited to, decalin, 1,2,3,7,8,8a-hexahydro-naphthalene, and the like. Tricyclic fused ring systems are exemplified by a cycloalkyl bicyclic fused ring system fused to an additional cycloalkyl group.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle” or “heterocyclyl” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

The term “substituted” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and, in particular, halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns, on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

The term “acid” refers to acids under both the Bronsted-Lowry and the Lewis definitions of acids. Under the Bronsted-Lowry definition, acids are defined as proton (H⁺) donors. Examples of Bronsted-Lowry acids include, but are not limited to, inorganic acids such as hydrofluoric, hydrochloric, hydrobromic, hydroiodic, perchloric, hypochlorous, sulfuric, sulfurous, sulfamic, phosphoric, phosphorous, nitric, nitrous, and the like; and organic acids such as formic, acetic, trifluoroacetic, p-toluenesulfonic, camphorsulfonic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, methanesulfonic, ethane disulfonic, oxalic, isethionic, and the like. Under the Lewis definition, an acid is an electron acceptor capable of accepting electron density by virtue of possessing unoccupied orbitals. Examples of Lewis acids include, but are not limited to, metal salts such as AlCl₃, FeCl₃, FeCl₃.SiO₂, CrCl₂, HgCl₂, CuCl, TiCl₄, Yb(OTf₃), InOTf, TiCl₂(OiPr)₂, and Ti(OiPr)₄; organometallic species such as trimethylaluminum and dimethylaluminum chloride; and boron species such as BH₃, B(Et)₃, BF₃, BF₃—OEt₂, BBr₃, B(OMe)₃, and B(OiPr)₃.

Examples of bases include, but are not limited to, alkali metal hydroxides, such as lithium hydroxide, sodium hydroxide, potassium hydroxide, and cesium hydroxide; alkali metal alkoxides, such as sodium methoxide, sodium ethoxide, sodium isopropoxide, sodium tert-butoxide, potassium tert-butoxide, lithium methoxide; alkali metal hydrides, such as lithium hydride, sodium hydride, and potassium hydride; alkali metal bicarbonates and carbonates, such as sodium bicarbonate, sodium carbonate, lithium bicarbonate, lithium carbonate, potassium carbonate, potassium bicarbonate, cesium carbonate, and cesium bicarbonate; organolithium bases, such as methyllithium, n-butyllithium, s-butyllithium, tert-butyllithium, isobutyllithium, phenyllithium, ethyllithium, n-hexyllithium, and isopropyllithium; amide bases, such as lithium amide, sodium amide, potassium amide, lithium hexamethyldisilazide, sodium hexamethyldisilazide, potassium hexamethyldisilazide, lithium diisopropylamide, lithium diethylamide, lithium dicyclohexylamide, and lithium 2,2,6,6-tetramethylpiperidide; and amine bases, such as pyridine, 4-(dimethylamino)pyridine, trimethylamine, diethylamine, triethylamine, diisopropylethylamine, 1,8-diazabicyclo[5.4.0]undec-7-ene (DBU), 1,5-diazabicyclo[4.3.0]non-5-ene (DBN), 1,4-diazabicyclo[2.2.2]octane (DABCO), and the like.

### Experimental Details

### Introduction

With this question in mind, we began our investigation by exploiting the high-resolution capabilities of a variant of functional magnetic resonance imaging (fMRI) to generate functional maps of the medulla in PD patients and controls. In contrast to other functional imaging techniques, which map glucose uptake, cerebral blood flow, or deoxyhemoglobin content, the technique used here maps steady-state cerebral blood volume (CBV) (Lin, W. et al. 1999; Moreno, H. et al. 2007). Although all these functional imaging variables are correlated, and all estimate regional brain metabolism (Small, S. A. 2003b), the main advantage of steady-state CBV is that it can generate functional brain maps with submillimeter spatial resolution (Lin, W. et al. 1999), a feature required for detecting dysfunction in small regions of the brain (Moreno, H. et al. 2007).

By comparing PD patients to controls, CBV maps of the medulla suggested that the DMNV is indeed dysfunctional in the disease. Furthermore, CBV maps identified a neighboring region, the inferior olivary nucleus (ION), as relatively unaffected by the disease, and guided by these imaging findings we generated gene expression profiles of the DMNV and ION harvested from postmortem brains with and without PD.

As previously described (Lewandowski et al. 2005), experimental studies in cellular and animal models or genetic analysis of human patients, are required to strengthen the mechanistic link between microarray findings generated from postmortem tissue and the disease under investigation. Among the findings that emerged from our microarray analysis, we decided to focus our investigation on an observed decrease in the expression of spermidine/spermine N1-acetyltransferase 1 (SAT1). SAT1 is the rate limiting catabolic enzyme in the polyamine pathway (Marton, L. J. & Pegg, A. E. 1995), and a decrease in SAT1 expression results in increased levels of higher order polyamines, spermine and spermidine. Notably, previous studies have suggested that polyamines increase α-synuclein aggregation (Antony, T. et al. 2003 and Gomes-Trolin, C. et al. 2002) in in vitro systems. Thus, additional studies in yeast models, mouse models, and human genetics were performed to confirm the pathogenic relevance of the polyamine pathway to PD and, more specifically, to suggest that increased levels of polyamines can accelerate α-synuclein pathology.

### Example 1

### Functional Imaging Identifies Brainstem Regions Targeted by and Resistant to Parkinson's Disease

### Materials and Methods:

### Brain Imaging Studies

Subjects: Subjects were recruited from a cohort of patients seen and rated by Dr. Lucien Coté at Columbia University Medical Center, Department of Neurology. At the time of the scan all patients were in early stages, rated at stage 1 or 2 according to the clinical criteria outlined in Hoehn and Yahr's Staging of Parkinson's Disease, Unified Parkinson Disease Rating Scale (UPDRS). Age matched subjects were acquired for the control group. Two of five PD and three of five controls were women. All controls underwent a neurological evaluation prior to the scans to assure their “control” status.

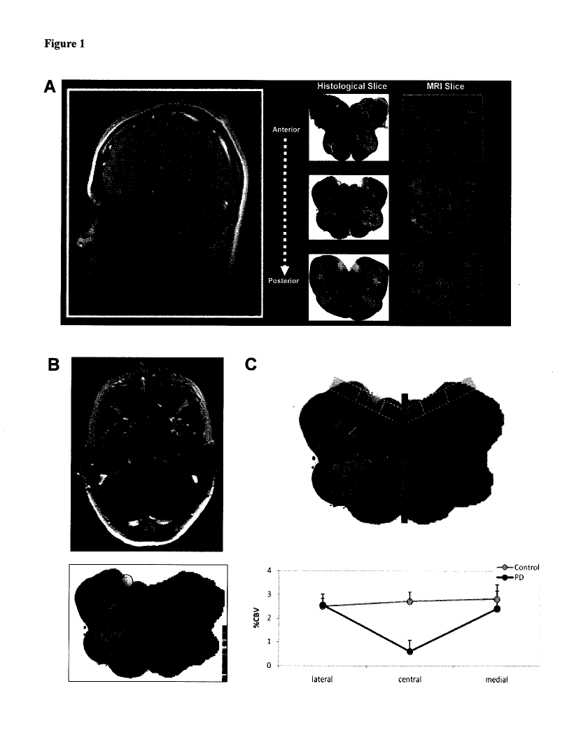

Data acquisition and processing: Subjects were imaged with a 1.5 tesla scanner Philips Intera scanner following a similar protocol as described (Lin, W. at al. 1999 and Moreno, H. et al. 2007). For each subject, 2 sets of oblique axial 3D T1-weighted images (TR=20 ms; TE=6 ms; flip angle=25 degrees; in plane resolution=0.86 mm×0.86 mm; slice thickness=2 mm) were acquired perpendicular to the long-axis of the brainstem (FIG. 1A). The first series of images were acquired before and the second acquired 4 minutes after IV administration of a standard dose of gadolinium-pentate (Omiscan, 0.1 mmol/kg).

Images were then transferred to a workstation containing an analysis software package (MEDx Sensor Systems, Sterling Va.). An investigator blind to subject grouping performed all imaging processing. The relatively short acquisition time minimized head motion, however the AIR program was used to co-register the images. A Gnu plot was generated to assess the quality of the co-registration, and an individual study was rejected if a shift greater than 1 pixel dimension was detected.

Among the series of anatomical (i.e, pre-contrast T1-weighted) images, fixed criteria based on the external morphology of the slices were used to identify the single slice in each individual that contained the DMNV. The external morphology of the medulla is dominated by upper quadrant protrusions, reflecting the inferior cerebellar peduncles, and lower quadrant protrusions, the inferior olivary nuclei. Moving from anterior to posterior, the target slice was always the one in which the anatomy of the medulla transitioned from a triangular to a box-like morphology, reflecting an inward shift in the inferior cerebellar peduncles (FIG. 1A). Moving from posterior to anterior, the target slice was always the one in which protrusions of the inferior olivary nuclei became visible.

Steady-state CBV maps derived from the contrast-induced changes in T1-weighted signal were generated as previously described (Lin. W. et al. 1999 and Moreno, H. et al. 2007). Briefly, subtraction maps were generated by subtracting the pre-contrast from the post-contrast image. Then, % CBV maps are generated by dividing the subtraction maps by a subtraction value measured from a region that is 100% blood, and multiplying by 100. In contrast to previous studies, that used a value measured from the sagital sinus (Lin, W. et al. 1999 and Moreno, H. et al. 2007), we used a value measure from the sigmoid sinus (FIG. 1B. upper image) because of its close proximity to the brain tissue of interest.

Data Analysis: a. Hypothesis-Driven Analysis:

In the absence of clear anatomical landmarks, regions of interest (ROIs) cannot be simply drawn over the DMNV, or neighboring dorsal nuclei. An alternative approach was used. A tracing tool in the imaging software program was used to manually trace the midline of the medulla, originating from the anterior median fissure extending to the median sulcus of the forth ventricle (FIG. 1C, upper image). From this midline, rectangular strips were drawn, 9 pixels in length and 3 pixels in width, that began from the lateral aspect of the dorsal medulla and extended to the midline (FIG. 1C upper image). Then, each rectangular strip was divided into 3 equal segments—lateral, central, and medial—with the central segment overlying the DMNV (FIG. 1C, upper image). These ROIs were then co-registered onto the CBV maps, yielding three CBV values per subject by combining measures on the right and left sides into mean values (FIG. 1C, lower graph).

A repeated measures ANOVA was constructed to test the hypothesis that the DMNV ROI was differentially affected in PD cases versus controls. CBV values from the 3 dorsal medulla ROIs were included as the within subject factors and diagnosis (PD vs. control) was included as the between subject factor.

b. Unbiased analysis: All CBV maps of the target slice were spatially filtered and co-registered, and on a pixel-by-pixel basis a t-test was used comparing the grouped PD images to the grouped control images. The yielded z-score was then colorwashed onto the standard anatomical image for visual inspection (FIG. 1B, lower image).

### Post-Mortem Studies

Gene-expression profiling: Parkinson's disease (PD) n=12 (6 DMNV and 6 ION) and control brain samples n=10 (5 DMNV and 5 ION) were obtained from the New York Brain Bank (NYBB) that were snap-frozen in liquid nitrogen and stored at −80° C. All PD cases were confirmed to have α-synuclein positive cytoplasmic inclusions.

The postmortem PD cases used displayed the pathological changes (Lewy body-containing neurons and Lewy neurites evidenced with antibodies directed against α-synuclein aggregates) that matched the pattern proposed by Braak (Braak, H. et al. 2003). Outsider cases were excluded. The clinico-pathological correlation of the cases of interest further confirmed the diagnosis of idiopathic PD. The thorough neuropathological evaluation performed ruled out the presence of changes that could have caused parkinsonism (e.g. neuronal and glial tangles as seen in progressive supranuclear palsy; or the synucleinopathic changes as observed in multiple system atrophy; or multiple infarcts centered within the striatum; or ubiquitinated, nuclear inclusions as seen in fragile X-associated tremor/ataxia syndrome, which mimics PD clinically). Lewy body-containing neurons or Lewy neurites (most of the time, both) are almost always found within the reticular formation of the medulla oblongata, and this in addition to the involvement of the dorsal motor nucleus of the vagus. Control brains were evaluated with the same protocol as the one used for the evaluation of the brains from patients. Control brains were identified without diagnostic abnormality. Within each brain the DMNV and ION were identified and sectioned using strict anatomical criteria following NYBB procedures.

Total RNA was extracted from each of the 22 tissue samples with TRIzol reagent (Invitrogen, Carlsbad, Calif.) and purified with RNeasy mini columns (Qiagen, Valencia, Calif.). All subsequent steps followed, Affymetrix's Eukaryotic Target Preparation Protocol found in the GeneChip Expression Analysis Technical Manual (www.affymetrix.com/support/technical/manual/expression_manual.affx available from Affymetrix). Also, all incubations were performed in a thermocycler (Eppendorf Mastercycler) to allow for proper temperature distribution during workup. A preparation of Poly-A RNA controls for two-cycle cDNA synthesis was made using an eukaryotic poly-A RNA control kit (Affymetrix). These poly-A spike-in controls were added to the total RNA that was isolated and used to prepare double-stranded cDNA. The T7-Oligo(dT) primer for cDNA synthesis was added and the first-cycle first strand master mix was added to the samples (Two-cycle cDNA synthesis kit, Affymetrix). This same kit was used to prepare the first-cycle second strand master mix. First-cycle IVT amplification of cRNA was performed on all samples using MEGAscript T7 kit (Applied Biosciences). First-cycle cRNA was cleaned up using a sample clean up module (supplied through Affymetrix). The cleaned cRNA was added to random primers and to the second-cycle first-strand master mix (using the Two-cycle cDNA synthesis kit) to produce cDNA. After incubation RNase H was added to each sample. T7-Oligo(dT) primer was added and then the second-cycle second-strand master mix. Following incubation T4 DNA polymerase was added. A sample cleanup module was used to clean up the double-stranded cDNA (Affymetrix). Using the GeneChip IVT labeling kit (Affymetrix) biotin-labeled cRNA was synthesized. The cRNA was cleaned and fragmented using the sample cleanup module (Affymetrix). Fragmenting was customized to a 100 Array Format. In the Gene Chip Analysis Facility of Columbia University, HG-U133A 2.0 microarrays (GeneChip, Affymetrix) were hybridized with fragmented cRNA for 16 h in a 45° C. incubator with constant rotation at 60 g. Microarrays were washed and stained on a fluidics station, and scanned using a laser confocal microscope. HG-U133A 2.0 microarrays were analyzed with Affymetrix Microarray Suite v5.0 and GeneSpring v5.0.3 (Silicon Genetics) software. Transcripts whose detection levels had a p-value greater than 0.05 were excluded and raw data from the 18,400 included transcripts were found on the included CD-ROM (GeneChip human genome U133A 2.0 Array Library File). Microarray data was processed and analyzed, as described (Lewandowski, N. et al. 2005). The microarray data has been submitted in a MIAME-compliant format to Gene Expression Omnibus (GEO) via NCBI (www.ncbi.nih.gov/geo), series accession #GSE11897.

Western blot analysis: A new set of 5 PD and 5 control brain samples were obtained from the NYBB, following the criteria described in SI Text (section: Gene expression profiling, PD pathology). Within each brain the DMNV and ION were identified and sectioned using strict anatomical criteria. Tissue samples were prepared as previously described (Small, S. et al. 2005). Specifically, tissue samples were soaked in five volumes of solution (0.32M sucrose, 0.5 mM CaCl₂, 1 mM MgCl₂, 1 mM NaHCO₃) supplemented with protease inhibitor cocktail (Roche) for 15 to 30 minutes. Samples were homogenized on ice with 12 strokes at 900 rpm using a motor operated Tephlon-pestle homogenizer. Homogenate was centrifuged at 240 g for 10 minutes at 4° C., and the supernatant was saved. Western blotting was performed on 25 μg of protein sample. Blots were incubated sequentially in Tris-buffered saline with tween 20 (TBS-T) for 1 hour, the mouse polyclonal anti-SAT1 (Novus Biologicals) was incubated overnight, and the appropriate enzyme horseradish persoxidase labeled secondary antibody was incubated for 1 hour and then washed with TBS-T and ECL reagent (GE Healthcare Amersham). Procedure was repeated using actin to normalize the samples.

Immunohistochemistry: Axial blocks of human medulla oblongata were frozen-sectioned using a Microm cryostat at 8 μm thickness as previously described (Small, s. et al. 2005). Specifically, tissue was directly quick-frozen onto the slides and postfixed with 4% paraformaldehyde in PBS, washed with PBS, and then treated with 3% H₂O₂, washed and preincubated for 1 hour in block solution consisting of 2% horse serum (Vector Laboratories), 1% bovine serum albumin (Sigma-Aldrich), and 0.1% Triton X-100 (Sigma-Aldrich) in PBS. Slides then were incubated 18 hours at 4° C. in diluted (1:75 in block solution) polyclonal antiserum to SAT1 (Proteintech Group Inc.). After washing with PBS, immunoreactivity was detected by an avidin-biotin-linked peroxidase method, using successive incubations and washes with anti-rabbit biotinylated IgG, Vectastain ABC-Elite reagent (Vector Laboratories), and diaminobenzidine (Sigma-Aldrich) chromogen reagent. Sections were washed with PBS, dehydrated, and mounted using Permount (Fisher Scientific).

### Results:

### Functional Imaging Identifies Brainstem Regions Targeted by and Resistant to PD

To determine whether the DMNV is dysfunctional in PD, CBV maps of the medulla, acquired perpendicular to the tong-axis of the brainstem, were generated in 5 PD patients (mean age=56.4) and 5 healthy age matched controls (mean age=56.2). For each individual subject, anatomical criteria were used to identify a single slice that contained regions of the dorsal medulla in which α-synuclein inclusions have been observed in early stages of PD pathology, in particular the DMNV (FIG. 1A and see methods). CBV is a fMRI variable with high submillimeter spatial resolution used to localize the DMNV and other subregions of the medulla.

CBV maps were spatially co-registered and an unbiased pixel-based analysis revealed a selective disease-associated CBV decrease in the general locale of the DMNV (FIG. 1B, lower image), while other areas of the medulla showed no difference between controls and PD patients. To further confirm this finding in a hypothesis driven manner, three regions-of-interest (ROIs) were generated over the dorsal medulla, with the “central” ROI overlying the DMNV (FIG. 1C, upper image). A repeated measures ANOVA revealed a significant group by region interaction (F=7.0; p=0.011) driven by a selective disease associated CBV decrease in the central ROI (FIG. 1C, lower graph). These results suggest that the DMNV is dysfunctional in PD.

### Imaging-Guided Microarray Identifies a PD Associated Decrease in SAT1 Expression

Relying on these imaging findings, we harvested the DMNV from 6 postmortem brains with evidence of PD and 5 control brains. The postmortem PD cases were evaluated for pathological changes (Lewy body-containing neurons and Lewy neurites evidenced with antibodies directed against α-synuclein aggregates) that matched the pattern proposed by Braak (Braak, H. et al. 2003). Further neuropathological evaluation was performed to confirm the diagnosis of PD over related diseases, (see methods).

We relied on the imaging results to identify a neighboring medullary region relatively unaffected by the disease to be used as a within-brain control. We decided on the inferior olivary nucleus (ION), because it is histologically identifiable, and harvested the ION from each of the 6 PD cases and 5 controls. Microarray techniques were used to generate gene expression profiles for each of the 22 tissue samples.

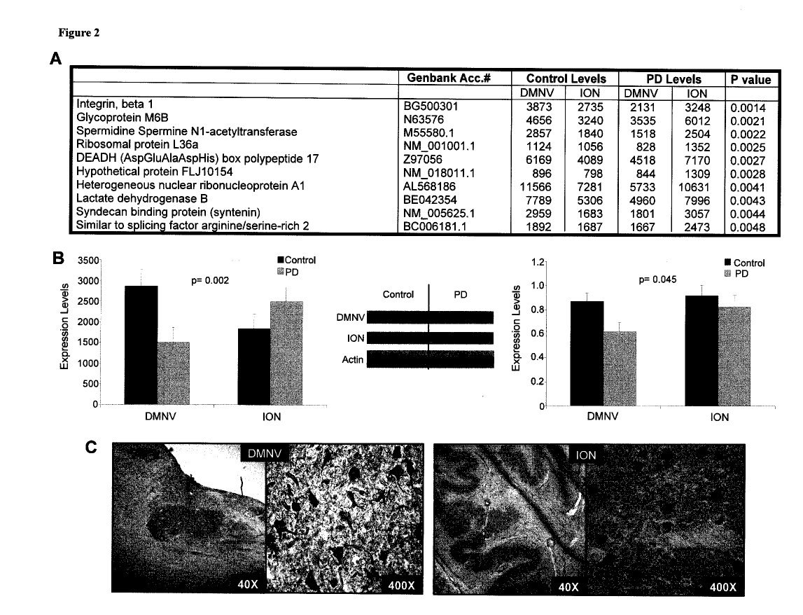

A repeated-measures 2×2 factorial ANOVA model constructed for the imaging study was applied to the expression dataset, in which expression levels from two regions of the medulla (DMNV vs. ION) were included as the within group factor, diagnosis (PD vs. controls) was the between group factor, and age and sex were included as covariates. Results revealed 10 transcripts whose expression levels showed a significant diagnosis by region interaction below a p value of 0.005. These 10 transcripts were all down-regulated in PD vs. controls (FIG. 2A).

Additional studies in cell culture, animal models, or human genetics are required to validate and interpret microarray findings identified in postmortem human brains (Lewandowski, N. et al. 2005). Informed by previous studies (Antonty, T. et al. 2003 and Gomes-Trolin, C. et al. 2002), we decided that among the transcripts that emerged from our microarray study further investigation was warranted for Spermidine/spermine N1-acetyltransferase 1 (SAT1), an enzyme that catalyzes the acetylation of polyamines spermidine and spermine. Because decreases in mRNA do not always indicate a decrease in protein levels (Chen, G. et al. 2002), we harvested the DMNV and ION from a new set of postmortem PD and control brains, and measured both SAT1 and actin by Western blot analysis. Replicating and extending the microarray finding (FIG. 2B, left bar graph), results revealed a PD specific decrease in SAT1 protein, normalized to actin, in DMNV (p=0.045) but not in the ION (p=0.19) (FIG. 2B, blot and right bar graph). Because α-synuclein inclusions are found predominately in neurons, we used immunohistochemistry to show that SAT1 is expressed in neurons of both the DMNV and the ION (FIG. 2C).

### Discussion:

To date, uncovering pathogenic molecular pathways associated with neurodegeneration has relied primarily on biochemical analysis of protein aggregates—identifying, for example, α-synuclein in Lewy bodies or Aβ peptide in amyloid plaques—or on linkage analysis to isolate genetic mutations. These two approaches have been important in clarifying the molecular biology of neurodegeneration (Litvan, I. et al. 2007), and have been successful in identifying pathogenic molecules underlying rare, monogenic, forms of disease. The introduction of gene expression profiling techniques, like microarray, has allowed the focus of molecular discovery to shift from aggregates and genes to brain cells themselves. Reflecting an interaction among multiple genetic and epigenetic factors, expression profiles of affected cells are, in principle, well suited for uncovering pathogenic pathways underlying complex disorders of the brain (Lewandowski, N. et al. 2005).

Nonetheless, microarray studies of neurodegenerative disease present a number of analytic challenges ((Lewandowski, N. et al. 2005). Because the earliest stages of the disease are characterized by neuronal dysfunction before cell death, and relatively small expression differences are sufficient to affect neuronal integrity, a microarray analysis applied to neurodegeneration must assume that meaningful expression differences might have relatively low effect sizes. Thus, in contrast to microarray applied, for example, to neoplasm, high signal amplitude cannot be relied on to filter out false positivity. Besides low signal amplitude, high signal noise is another analytic limitation of microarray applied to neurodegeneration. In particular, inter-individual differences in the dying process have a strong affect on gene expression levels (Li, J. Z. et al. 2004), independent of the disease under investigation, and this source of noise needs to be factored out when tissue samples are harvested from postmortem brains.

These analytic challenges can be partially addressed by relying on prior imaging information to construct a hypothesis driven model predicting how a pathogenic molecule anatomically targets a brain structure (Lewandowski, N. et al. 2005 and Pierce, A. et al. 2004). As in other neurodegenerative diseases, PD is assumed to cause “cell sickness” before cell death, and the relationship between histological markers and neuronal dysfunction is undetermined (Dauer, W. et al. 1992). Functional imaging, therefore, is well suited to establish an anatomical pattern of disease. Until recently, however, most functional imaging techniques did not possess sufficient spatial resolution to map dysfunction in the small regions of the medulla. Over the last few years, with a primary interest investigating the small regions of the hippocampal formation, we have optimized a variant of fMRI notable for its extremely high submillimeter spatial resolution (Moreno, H. et al. 2007 and Schobel et al. 2009).

Our imaging findings document, for the first time, that PD patients have dysfunction in the dorsal motor nucleus of the vagus. Notable for a relative absence of cell death and gliosis, regions of the medulla are particularly well suited for microarray experiments because expression profiles are less likely to be confounded by differences that reflect dying cells or activated astrocytes. Thus, identifying regions in the medulla differentially affected by and resistant to PD was the main purpose of our imaging study. Future large scale imaging studies, in which brainstem CBV maps are generated in a larger number of PD patients across different stages of the disease, are required to determine whether observed brainstem dysfunction correlates with signs and symptoms of the disease, whether it antedates dysfunction in other brain regions, and whether it can be used for diagnostic purposes.

In our second study, imaging-guided microarray revealed a limited set of mRNA transcripts differentially affected in the DMNV of PD brains. Although the cellular function of a number of these transcripts are interesting, arriving at mechanistic or causal interpretations are difficult based on findings from postmortem brains alone (Lewandowski, N. et al. 2005). As in all neurodegeneration, the disease process in PD exists many years before the patient's death and it is impossible to know whether a postmortem finding reflects an upstream defect that affects α-synuclein toxicity or a downstream effect reflecting protracted neuronal dysfunction. Thus, additional studies are required to interpret the mechanistic and causal role of microarray findings identified in postmortem samples (Lewandowski, N. et al. 2005). For example, in our previous studies, imaging-guided microarray applied to Alzheimer's disease identified deficiencies in the retromer sorting pathway (Small, S. et al. 2005). Only by systematically manipulating retromer-related molecules, however, in cell culture (Small, S. et al. 2005), animal models (Muhammad, A. et al. 2008), and by linking genetic variance to the disease in human patients (Rogaeva, E. et al. 2007), was retromer sorting validated as a pathogenic pathway (Small, S. A. 2008).

Among the transcripts identified in the microarray study, we decided to focus our attention on the observed decrease in SAT1 expression. SAT1 is the rate limiting enzyme in polyamine catabolism, and a reduction in SAT1 leads to an increase in higher order polyamines, in particular spermidine and spermine (Libby, P. R. and Porter, C. w. 1992; Niiranene, K. et al. 2006). A previous study showed that PD patients have elevated levels of spermidine and spermine in red blood cells (Gomes-Trolin, C. et al. 2002), which do not themselves produce polyamines. Another study showed that in an in vitro system, exogenously added spermidine and spermine accelerate the aggregation of e-synuclein (Antony, T. et al. 2003). Furthermore, previous studies have shown that SAT1 is expressed in numerous brain regions implicated in PD, including the substantia nigra pars compacta (Zoli, M. et al. 1996).

### Example Two

### Yeast Studies Establish a Link Between Polyamines and A-Synuclein Toxicity

### Materials and Methods:

### Yeast Studies

Yeast Strains and Media: All yeast studies used the W303 strain (MATa ade2-1 can1-100 his3-11,15 leu2-3112,trp1-1, ura3-1). The α-synuclein (α-syn) strains used for growth rate assays were as follows: a control strain, containing an empty vector (pRS304) at the TRP1 locus, a strain expressing wildtype human α-synuclein fused to GFP at the TRP1 locus (pRS304Gal-α-SynWT-GFP), and a strain expressing human α-synuclein (A53T)-GFP fusion protein at the TRP1 locus (pRS304Gal-α-SynA53T-GFP). The α-syn overexpressing yeast strain used for Tpo4 characterization was W303 with α-syn integrated into HIS3 and TRP1 loci (IntTox): pRS303Gal-αSynWT-YFP pRS304Gal-αSynWT-YFP. Strains were manipulated and media prepared using standard techniques. The A53T mutation enhances the toxicity of α-synuclein both in vitro (Conway, K. A. et al. 1998) and in yeast (Outeiro, T. F. et al. 2003), and is associated with the autosomal dominant form of familial Parkinson's disease in man (Polymeropoulos, M. H. et al. 1997).

Growth Rate Assays: Freshly growing yeast cells were diluted to OD600 0.1 in YPGal medium (1% yeast extract, 2% peptone, and 2% galactose). Growth curves were generated by growing cells in YPGal medium supplemented with different concentrations of spermine at 30° C. OD600 was monitored at a one hour interval using the Bioscreen C (Growth Curves USA, Piscataway, N.J.).

### Yeast Screen:

Tpo 4 was identified in an unbiased genome wide screen investigating modifiers of αSyn toxicity (Yeger-Lotem, E. et al. 2009). Briefly, 5000 yeast open reading frames were cloned individually into a plasmid with a galactose inducible promoter, pBY0111 (CEN, URA3, AmpR). Cells expressing IntTox aSyn were transformed with the clones and grown 2 days on synthetic drop out media. The cells were then spotted onto SD-Ura or SGal-Ura plates. Modifiers of aSyn toxicity were identified after 3 days of growth on galactose plates. Tpo4 was retested three times for confirmation.

α-Syn Growth with Tpo4

α-Synuclein (α-syn) strains and control strains were grown overnight to saturation in glucose containing media. Cultures were normalized for their OD and serially diluted five-fold prior to spotting onto yeast media plates containing either glucose or galactose.

### α-Syn Microscopy

Cells were grown overnight in glucose containing media. Culture densities were normalized, washed with water, and induced for six hours in galactose. Cells were fixed (4% formaldehyde, 50 mM KPi pH=6.5, 1 mM MgC12) for one hour on ice, washed twice with PBS and imaged.

### Results:

A decrease in SAT1 increases levels of the polyamines spermidine and spermine and previous in vitro studies have suggested that polyamines increase the aggregation of α-synuclein (Antony, T. et al. 2003; Grabenauer, M. et al. 2008; Cooper, A. A. et al. 2006). To investigate the relationship between polyamines and α-synuclein in an in vivo system, we turned to an established yeast model of α-synuclein toxicity (Outeiro, T. F. et al. 2003). In this model yeast expressing one copy of α-synuclein grow normally (NoTox strain), cells expressing ˜40% higher levels grow slowly (IntTox), and cells expressing twice as much protein die (HiTox), recapitulating the extreme dosage sensitivity this protein displays in association with PD in humans (Gilter, A. D. et al. 2008).

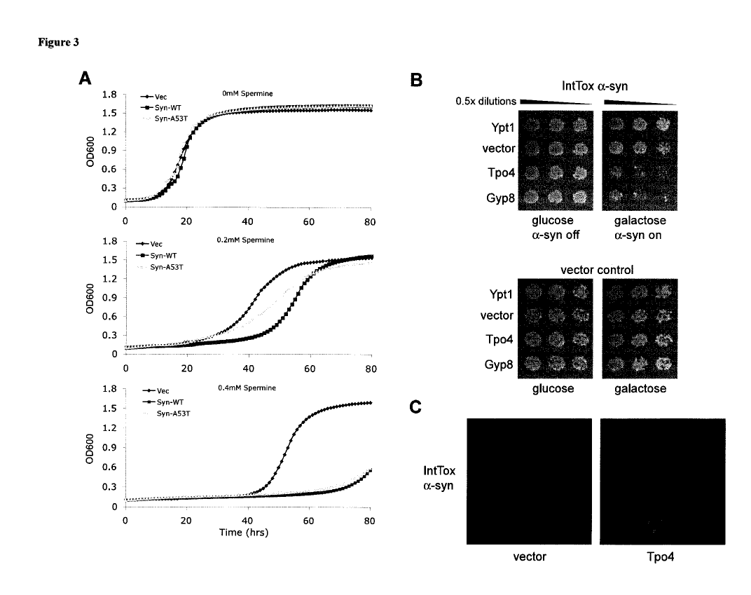

We examined the effects of exogenous spermine on yeast strains expressing either wildtype α-synuclein (Syn-WT) or the A53T α-synuclein mutant (Syn-A53T). This mutation enhances the toxicity of α-synuclein both in vitro (Conway, K. A. et al. 1998) and in yeast (Outeiro, T. F. et al. 2003), and is associated with the autosomal dominant form of familial Parkinson's disease in man (Polymeropoulos, M. H. et al. 1997). Spermine was found to be more toxic to yeast expressing α-synuclein (wildtype and A53T) than the strain carrying the empty vector (FIG. 3A), suggesting a relationship between polyamines and the cellular toxicity of α-synuclein.

IntTox, the strain with intermediate levels of α-synuclein expression, provides a sensitized background for detecting genes that enhance or suppress toxicity. When α-synuclein accumulates to toxic levels in this strain, one of the earliest detectable defects is a block in the trafficking of secretory vesicles from the endoplasmic reticulum to the Golgi. Proteins that promote forward vesicle trafficking (e.g. the Rab1 GTPase, Ypt1) suppress α-synuclein toxicity while those that inhibit it (e.g. the Rab1 GTPase activating protein, Gyp8) enhance it (Cooper, A. A. et al. 2006). In an unbiased genome-wide screen for modifiers of α-synuclein toxicity, we recovered Tpo4 as an enhancer of toxicity (Cooper, A. A. et al. 2006; Yeger-Lotem, E. et al 2009). Tpo4 is member of a family of proteins responsible for polyamine transport in S. cerevisiae (Tomitori, H. et al. 2001). Quantitative tests of Tpo4, relative to Ypt1 and Gyp8, confirmed the effect of this polyamine transporter in enhancing the toxicity of α-synuclein when overexpressed (FIG. 3B). Notably, in the IntTox strain overexpression of Tpo4 caused α-synuclein to form intracellular foci more rapidly than it otherwise would (FIG. 3C). These α-synuclein foci were previously demonstrated to occur at sites of stalled vesicles (Gilter, A. D. et al. 2008). Deletion of Tpo4 had no effect on α-synuclein toxicity, likely due to the redundancy of polyamine transporters in yeast (data not shown). Taken together, the data suggest connections between α-synuclein, polyamines, vesicle trafficking, and membrane metabolism.

### Discussion:

Whether polyamines can modify the cellular toxicity of α-synuclein in vivo was unknown. To address this issue, we turned to a yeast model of PD and showed in an unbiased screen that Tpo4, responsible for polyamine transport in S. cerevisiae, and exogenous spermine accelerated the toxicity of yeast expressing human α-synuclein. Importantly, because we are interested in sporadic PD, in which non-mutated forms of α-synuclein play a mechanistic role, acceleration of the phenotype was found in yeast expressing wildtype α-synuclein.

### Example Three

### Mice Studies Establish a Link Between SAT1 Activity and A-Synuclein Histopathology

### Materials and Methods:

### Transgenic Mice Studies

α-Synuclein transgenic mice and infusion: For this study, heterozygous transgenic (tg) mice (Line 61) expressing wildtype human α-synuclein (hα-syn) under the regulatory control of the mThy1 promoter were used (Rockenstein, E. et al. 2002). These animals were selected because they display abnormal accumulation of detergent-insoluble hα-syn and develop hα-syn-immunoreactive inclusion like structures in the basal ganglia, cortex, and limbic system (Rockentein, E. et al. 2002). Furthermore, these animals also display neurodegenerative and motor deficits that mimic certain aspects of PD and Dementia with Lewy bodies (Fleming, S. et al. 2004). Infusions were performed as previously described (Veinbergs, I. et al. 2001). In order to evaluate the effects of the polyamine pathway, hα-syn tg mice (10-11 months old) received intraventricular infusions with a cannula implanted into the skull and connected to osmotic minipumps of either vehicle, Beneril, or DENSPM. A total of 15 hu-syn tg mice (n=5 per group) were used for these experiments. Mice were treated for six weeks; the compounds were dissolved in 0.9% NaCl at a concentration of 62 μM for DENSPM and 30 μM for Berenil at a pH of 7.2. Then, 200 μL of these solutions were filled into the osmotic minipump (Alzet) ensuring constant delivery (0.25 μL/h). The minipump was implanted subcutaneously on the back under light anesthesia. An additional group of control non tg (n=5) mice were implanted with minipumps filled with vehicle only. All procedures were completed under the specifications set forth by the Institutional Animal Care and Use Committee.

### Immunohistochemical Analysis of α-Synuclein Accumulation and Neurodegeneration:

Brains were removed and divided sagittally. One hemibrain was postfixed in phosphate-buffered 4% paraformaldehyde, pH 7.4, at 4° C. for 48 h and sectioned at 40 μm with a Vibratome 2000 (Leica) and placed in cryosolution, whereas the other hemibrain was snap frozen and stored at −70° C. for further analysis. Sections were first washed 3×5 minutes each in PBS. They were then pretreated with 1% Triton-X, 3% hydrogen peroxide in PBS for 15 minutes, washed again 3×5 min in PBS and blocked in 10% serum matching the animal the secondary antibody was raised in for 1 hour and then washed in PBS. Sections were then placed in primary antibody: mouse anti-TH, rabbit anti-(h) α-syn, mouse anti-MAP2 (Chemicon, (1:500)) overnight in 4° C. Sections were washed 3×5 min in PBS and then placed in biotynilated secondary antibody (1:100) (Vector Laboratories) for 2 hours. After washing again 3 times in PBS, sections were placed in 20% diaminobenzene (DAB) (Vector Laboratories) for 20 seconds. The reaction with DAB was halted by immersing the sections in double distilled water. Sections were then mounted, dried and coverslipped with Entillin (Fisher Scientific). Sections for fluorescent immunohistochemistry were treated with mouse monoclonal anti-microtuble-associated protein 2 (MAP2, 1:50, Roche Molecular Biochemicals) primary antibody. This antibody was then detected with the fluorescein isothiocyanate (FITC)-conjugated anti-mouse secondary antibody (Vector Laboratories). Coverslips were air-dried overnight, mounted on slides with anti-fading media (Vectashield, Vector Laboratories), and imaged with the LSCM (MRC 1024, Bio-Rad). The immunolabeled blind-coded sections were imaged by LSCM (MRC1024, Bio-Rad) and analyzed with the Image 1.43 program (NIH), as previously described (Fleming, S. et al. 2004).

### Results:

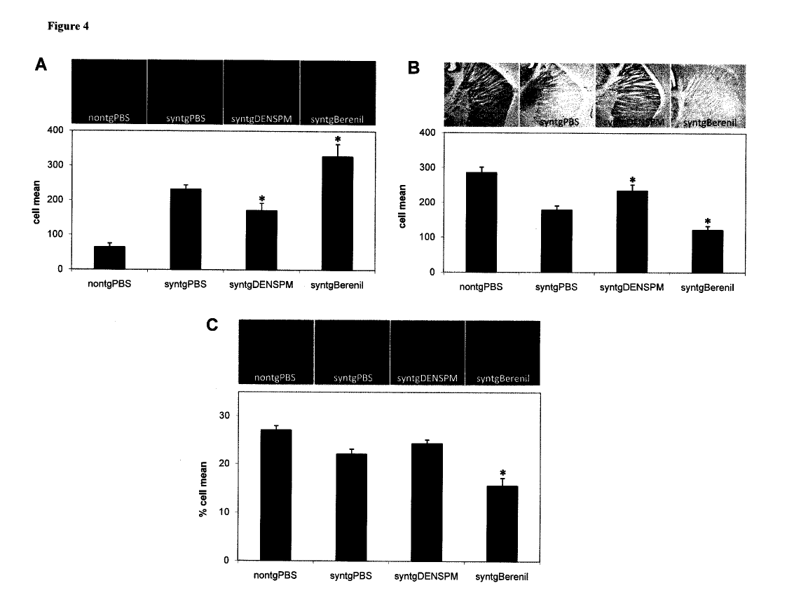

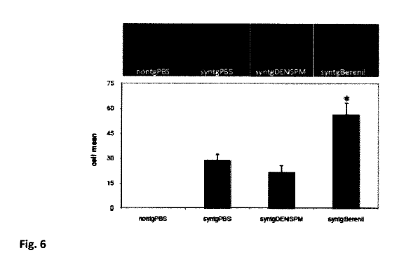

To investigate the relationship between SAT1 activity and α-synuclein histopathology in the mammalian brain we turned to genetically modified mice that express wildtype human α-synuclein and develop histological inclusions (Rockentein, E. et al. 2002). Berenil (diminazene aceturate) is a pharmacological agent that reduces SAT1 activity (Libby, P. R. & Porter, C. W. 1992), while DENSPM (N1, N11-diethylnorspermine) is a polyamine analog that increases SAT1 activity (Thomas, T et al. 2001). We chronically delivered these agents via osmotic minipumps to the transgenic mouse model. Results showed that, compared to the transgenic mice treated with PBS, neuronal accumulation of α-synuclein in the basal ganglia (FIG. 4A) and focused within the substania nigra (FIG. 6) was increased with Berenil (p=0.009) and decreased with DENSPM (p=0.043). Berenil worsened the effects on tyrosine hydroxylase (TH) fibers (p=0.033), while DENSPM partially rescued the deficits (p=0.022) (FIG. 4B). Alterations in the dendritic complexity in the basal ganglia of the α-synuclein transgenic mice were worsened by Berenil (p=0.013) and ameliorated by DENSPM, as detected by MAP2 (FIG. 4C). Therefore, overall α-synuclein pathology in these mice was worsened with Berenil and partially rescued with DENSPM. It should be noted that these drugs showed no significant effect in non transgenic mice (nontg) when compared to nontg treated with PBS, suggesting that the effect may be dependent on α-synuclein expression. However, it should be noted that general toxicity was associated with the route of administration. Infusion caused death before the termination of the experiment in some of the control and pharmacologically treated mice.

The next important question was whether SAT1 activity can modify α-synuclein histopathology in the mammalian brain. To address this question, we turned to a transgenic mouse model of PD that expressed wildtype human α-synuclein, and treated them with pharmacological agents, Berenil and DENSPM. Berenil (diminazene aceturate) has been shown to inhibit polyamine metabolism by reducing SAT1 activity via competitive inhibition of the enzyme (Rockenstein, E. et al. 2002). Berenil was favored above other SAT1 inhibitors due to its selectivity for effecting SAT1 alone, and no other enzymes involved in polyamine metabolism (Wallace, H. M. et al. 2004). DENSPM (N1, N11-diethylnorspermine) is a polyamine analog and the most potent known inducer of SAT1 activity. A number of studies have demonstrated DENSPM's ability to down-regulate polyamine biosynthetic enzyme activities and suppress transport of polyamines, as well as its potent upregulation of SAT1, resulting in depletion of intracellular polyamine pools (Porter, C. W. et al. 1992; Casero, R. A. et al. 1989). Using these pharmacological agents that mediate SAT1 activity, results showed that Berenil worsened the PD phenotype, while DENSPM partially improved the PD phenotype in these mice.

### Example Four

### Genetic Studies in Human Patients Provides Provisional Support Linking SAT1 to PD

### Materials and Methods:

### Genetic Studies

Patient population: The SAT1 gene was sequenced in 92 PD patients who participated in the Genetic Epidemiology of PD (GEPD) study (Marder, K. et al. 2003A; Marder, K. et al. 2003B; Levy, G. et al. 2004). An additional 389 cases and 408 controls from GEPD were genotyped for the SAT1 c.786_{—}788delTGT (NM_{—}002970) variant. Cases were ascertained from the Center for Parkinson's Disease and Other Movement Disorders at Columbia University based on age at onset of motor signs ≦50 (EOPD) or >50 (LOPD), with oversampling of the EOPD group (Marder, K. et al. 2003A). The majority of the controls were recruited by random digit dialing, with frequency matching by age, gender, and ethnicity. All participants were seen in person and underwent an identical evaluation (Marder, K. et al. 2003A) that included a medical history, Unified Parkinson's Disease Rating Scale (UPDRS) (Fahn, S. et al. 1987), and videotape assessment.

Sequencing and Genotyping: Polymerase chain reaction (PCR) and amplification of all exons and exon/intron boundaries of the SAT1 gene were performed. The PCR and sequencing primers used for amplification of SAT1 are available upon request. Cycle sequencing in forward and reverse directions was performed on purified PCR products and run on an ABI 3700 genetic analyzer (Applied Biosystems). Sequence chromatograms were viewed and genotypes determined using Sequencher (Genecodes).

Statistical Analysis: Demographic and clinical characteristics of PD cases compared to controls and SAT1 wildtype compared to SAT1 c.786_{—}788delTGT (NM_{—}002970) variant carriers were analyzed using chi-square tests or Fisher's exact tests for categorical data and student's t-tests for continuous data.

Protein, mRNA Levels, and Stability of Splice Variants:

a. Lymphocytes: RNA was extracted from cultures of lymphoblastoid cell lines established by transforming patients' lymphocytes with Epstein-Barr virus using standard techniques (Marder, K. et al. 2003B). Cells were homogenized and RNA extracted using TRIzol reagent (Invitrogen). RNA was further purified using the RNeasy Mini Kit (Qiagen) with on-column DNase digestion. cDNA was synthesized from 1 μg total RNA using Superscript III First Strand Synthesis Supermix (Invitrogen). The following TaqMan Gene Expression assays (Applied Biosystems) were used for real time-PCR relative quantification of mRNA levels: Hs00161511, Hs00971737, Human ACTB Endogenous Control, and Human GAPDH Endogenous Control. The reactions were performed in triplicate on an AB7500 Real Time System using TaqMan Universal PCR Master Mix (Applied Biosystems) according to the manufacturer's instructions. The data were normalized to the geometric mean of β-ACTIN and GAPDH and the 2^{-ΔΔCt} method was used to obtain relative expression values. Comparisons were made between lymphoblastoid cell lines for each SAT1 mRNA variant separately.

b. HEK 293 cells: HEK 293 cells were cultured in Dulbecco's modified Eagle's medium containing 10% FBS, 100 U/ml penicillin, 100 μg streptomycin, and 0.1 mM non-essential AAs (GIBCO, Invitrogen) in an atmosphere of 5% CO₂ at 37° C. Cells were seeded in six-well plates at approximately 5×10⁵ cells/well and transfections were performed the next day using 2.5 μg of construct (SAT1-s wt/del, SAT1-1-wt/del, GENEART) using Lipofectamine 2000 (Invitrogen). Each plasmid variant was cotransfected with a GFP plasmid (GeneScript), to control for transfection efficiency. A plate with each variant transfection was lysed for protein analysis via western blot (following methods described prior) using anti-mouse GFP antibody (GeneScript) and anti-rabbit SAT1 (Novus Biologicals), normalized to tubulin. After 48 hrs the remaining transfected cell plates were treated with actinomycin D (GBioSciences) and RNA was extracted at different time points (time=0, 2 hrs, 4 hrs, 6 hrs, 12 hrs, 24 hrs, and 48 hrs) for real time-PCR analysis as described for lymphoblasts. Data for each construct was normalized to t=0.

### Results:

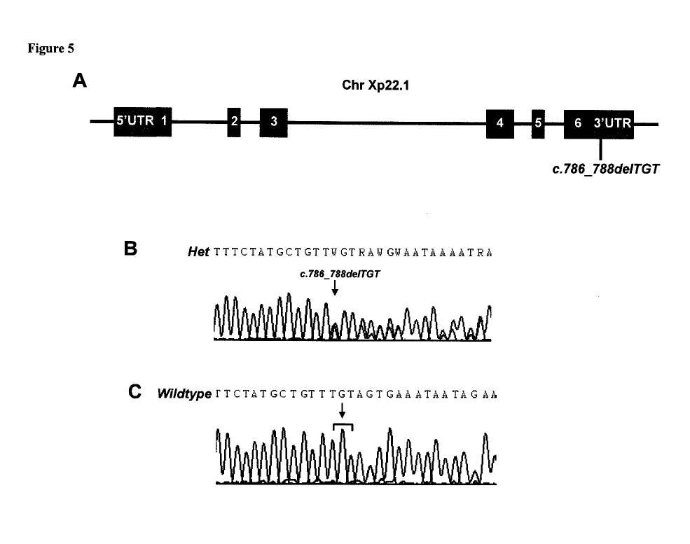

As a complex disorder we do not assume that defects in the SAT1 gene necessarily play a role in polyamine abnormalities in PD. Nonetheless, it was worth exploring this issue, as any genetic finding would strengthen the link between SAT1 and PD. Initially we sequenced all exons and adjacent exon/intron boundaries of the SAT1 gene in a total of 92 PD patients. We identified a novel variant, c.786_{—}788delTGT (NM_{—}002970), in the 3′UTR of the SAT1 gene in 2 PD patients (FIG. 5). One patient was heterozygous and one hemizygous for the c.786_{—}788delTGT variant. To evaluate the frequency of the 3′UTR variant we extended the analysis to genotyping of additional patients and controls enrolled in Genetic Epidemiology of PD, GEPD (total n=797 subjects; patients n=389 and controls n=408). Overall, a total of 4 PD patients were identified that carried the c.786_{—}788delTGT variant, including 3 heterozygotes and 1 hemizygote. The c.786_{—}788delTGT variant was absent in all 816 control chromosomes (n=408 controls). The mean age of the 4 PD patients carrying the c.786_{—}788delTGT variant was 63.3 (range 51-72), the mean age at onset was 54 years (range 38-68, with 2 with onset <50) and mean duration of PD was 9.25 years (SD 7.5). There were three women and one man, three white, and one “other” ethnicity. None reported a family history of PD in a first degree relative. Three of the four had tremor at rest as a first symptom, and all were levodopa responsive. None of the four were demented at the time of evaluation. None of the cases carried mutations in Parkin, LRRK2, or DJ1.

A number of studies were conducted to determine whether the identified genetic variant in SAT1 affects gene expression levels. SAT1 undergoes alternative splicing. The longer alternative spliced variant, SAT1-1, differs from the common form, SAT1-s, with the insertion of an additional exon between exons 3 and 4 with in-frame premature termination codons (Ichimur, S. et al. 2004). Both share the same 3′UTR. We used TaqMan gene expression assays to compare endogenous mRNA levels of both splice variants of SAT1 between lymphoblasts obtained from 1 male PD patient carrying the SAT1 c.786_{—}788delTGT variant in the 3′UTR of the gene and four non-carrier male PD patients. No significant differences in either of the splice variants were found in the PD patient carrying the SAT1 variant when compared with non-carrier PD patients.

To investigate this further, a second experiment was aimed at determining whether the deletion would affect mRNA stability. We measured mRNA from HEK 293 cells transfected with plasmids carrying either the SAT1 c.786_{—}788delTGT variant or wildtype SAT1-s or SAT1-1 at different time points after inhibiting transcription. No significant differences in mRNA degradation were found between the deletion carrying or wildtype mRNA in either splice variant. Cells from each transfected variant (wildtype: of SAT1-s & SAT1-1, and c.786_{—}788delTGT variant: of SAT1-s & SAT1-1) were also assayed for total SAT1 protein. No significant difference was seen amongst the variants and their deletion carrying vs. wildtype derivatives.

### Discussion:

Finally, we decided to sequence SAT1 in human patients, even though a genetic variant in this gene was not necessarily assumed. In fact, as a complex disorder, we believe that polyamine abnormalities observed in PD are more likely to reflect a complex interaction between genes, epigenetics, and the environment rather than defects in the SAT1 gene itself. However, because any genetic finding in human patients would validate the potential pathogenicity of a microarray finding, we reasoned that it was worth exploring this issue. Remarkably, a novel variant, c.786_{—}788delTGT, in the SAT1 gene was indeed found. It is important to emphasize that our molecular studies suggest that the c.786_{—}788delTGT does not clearly affect levels of gene expression. Furthermore this variant is rare, occurring in less than 1% of the patients examined, but the variant was exclusive to patients and absent in all 816 of the control chromosomes genotyped. Beyond the scope of the current body of work, future genetic studies genotyping a larger number of cases and controls are required to determine the exact frequency of c.786_{—}788delTGT, and future molecular studies will determine whether and how this variant affects the enzymatic function of SAT1. Despite these outstanding questions, the surprising disease associated mutation identified in SAT1 does, we believe, strengthen the pathogenic link between the polyamine pathway and PD. Future molecular studies, and future CBV studies—imaging patients who are found to carry the SAT1 c.786_{—}788delTGT variant—might identify a functional consequence of this variation.

In summary, the range of interleaved studies establishes that defects in the polyamine pathway play a role in PD pathogenesis. A number of pharmacological agents have already been developed that enhance the function of SAT1 (Gemer, E. W. et al. 2004). Based on our current findings, we have begun exploring whether any of these agents cross the blood-brain barrier, and if so plan on initiating drug studies, in conjunction with functional imaging, to determine whether reductions of brain polyamine levels ameliorate this common and under treated disease.

### Example Five

### Synthesis and Production of DFMO Analogs

The structure for new DFMO Analogs. DFMO Analog 1, DFMO Analog 2, DFMO Analog 3 and DFMO Analog 4 is provided below. DFMO Analog 1 (C₅H₁₆F₂N₂O₂) has an Exact Mass of 210.12 and DFMO Analog 2 (C₁₄H₂₄F₂N₂O₆) has an exact mass of 354.16. The synthesis for DFMO Analog 1, DFMO Analog 2, DEMO Analog 3 is also provided.

Structure of DFMO and DFMO Analogs:

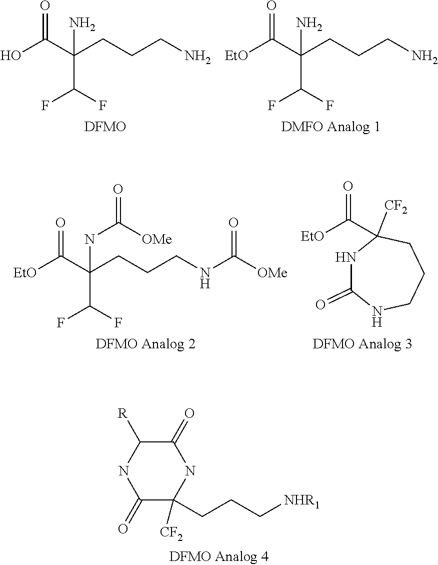

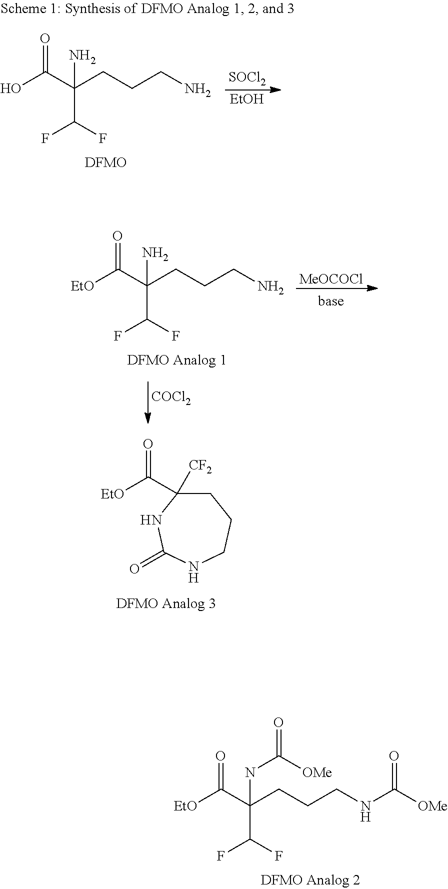

### Example 6

### Prevention of DFMO Cytostasis

### Materials and Methods

Cell Growth. Murine L1210 leukemia cells were maintained in loga rithmic growth as a suspension culture in RPMI-1640 medium containing 2% 4-(2-hydroxyethyl)-1-piperazineethanesulfonic acid-3-(W-morpholino) propanesulfonic acid, 1 HIM aminoguanidine, and 10% NuSerum (Collaborative Research, Inc., Lexington, Mass.). Cells were grown in either glass culture tubes in a total volume of 2 ml or 25- or 75-sq cm tissue culture flasks in a total volume of 15 and 50 ml, respectively, under a humidified 5% CO2 atmosphere at 37Â° C. Cultures were treated while in logarithmic growth (0.5 to 1×105 cells/ml) with the SPD derivatives, or SPD at 0.1 mM. After 24 or 48 h, cells were removed from tubes for counting and viability determinations. Cell number was determined by electronic particle counting (Model 2F Coulter Counter; Coulter Electron-ics, Hialeah, Fla.) and confirmed periodically with hemocytometer measurements. Cell viability was assessed by trypan blue dye exclusion (0.5% in unbuffered 0.9% NaCI solution). The percentage of control growth was determined as follows.

% of control growth=Final treated cell no.−initial inoculum(5×10*cells/ml)Final untreated cell no.−initial inoculums×100

Only viable cells were considered in this calculation. Derivatives active at 0.1 mM were further evaluated at lower concentrations for ICÂ>> determinations.

### Prevention of DFMO Cytostasis.

The ability of SPD or the various SPD derivatives to affect DFMO-induced cytostasis was studied by exposing cells to 5 mM DFMO plus 10/IM SPD or a SPD derivative for 48 h. Cell growth and percentage of inhibition of growth were determined and compared to those values achieved with DFMO alone and DFMO plus SPD.

### Polyamine Determinations

Cell samples were washed twice in cold PBS, and an aliquot of 107 cells was removed for polyamine determination. The cells were pelleted, and the PBS supernatant was carefully removed with a cotton swab. The remaining cell pellet was extracted with 0.5 ml of 0.6M perchloric acid for 30 min at 4° C. and then centrifuged for 3 min at 12,000×g using a microcentrifuge. The supernatant was frozen at −20° C. until analysis by HPLC.

### Polyamine Extraction Method:

Cultured cells
↓washed twice in cold PBS
↓Centrifuge
Cell pellet
↓Extracted with 0.5 ml of 0.6M Perchloric acid at 4° C. for 30 min
↓Centrifuge at 12,000×g for 3 min
Supernatant (stored at −20° C.)

Polyamines in a 50-̂1 sample of perchloric acid extract were separated on an HPLC system using a 2.8-mm-diameter glass microbore column with a 2-cm column height packed with DC-4A cationic exchange resin (Durrum Chemical Corp., Palo Alto, Calif.). The column temperature was maintained at 65° C. with water from a circulating bath. The column was eluted at a flow rate of 16 ml/h with an initial column pressure of 500 psi which decreased with increasing ionic strength of the elution buffer. Buffer 1 containing 0.2 M boric acid, 0.5 M NaCI, 0.03% Brij 35 (Pierce Chemical Co., Rockford, Ill.), and 0.0001% octanoic acid (pH adjusted to 6.0 with saturated KOH) was run through the column for 4 min. Buffer 2 containing 0.2 M boric acid, 2.15 M NaCl, 0.03% Brij 35, and 0.0001% octanoic acid (pH 6.0 as above) was run for 6 min. Buffer 3 containing 0.2 M boric acid, 2.9 M NaCI, and 0.0001% octanoic acid (pH 6.0 as above) was run for 6 min. The column was reequilibrated for 10 min with Buffer 1 prior to loading the next sample. The column eluate was derivatized with 0.05% o-phthalaldehyde (Durrum Chemical Corp.) in 0.4M borate buffer (pH 10.4) containing 1 HIM2-mercaptoethanol and 0.09% Brij 35. The o-phthalaldehyde reagent flow rate was 8 ml/h. The derivatized eluate was analyzed for polyamine content by passing it through the flow cell of a flowmeter (Fluoro-Monitor; American Instrument Co., Silver Spring, Md.) with a fixed excitation wavelength of 360 nm and emission wavelength of 570 nm. Data were analyzed using a Hewlett Packard Model 3385A automation system. The system variance for a standard containing known quantities of PUT (putrescine), SPD (spermidine), and SPM (spermine) hydrochloride is less than 5%. The sensitivity of the HPLC system is in the range of 50 pmol/50-M1 sample (106 cells). Run time was less than 20 min per sample plus 10 min for column equilibration.

### Dilution of Standard Curves

Dilution of Standard Polyamines: Spermine
and Spermidine for Calibration Curve
Spermidine: Sigma 4139-1G
Conc: 925 mg/ml

| | | | Volume | Solvent | | |
|---|---|---|---|---|---|---|
| | Stock | | (μl) | (μl) | Final | |
| 1 | 925 | mg/ml | 10.81 | 989.19 | 10 μg/ml (=10,000 ng/ml) | |
| 2 | 10 | μg/ml | 100 | 900 | 1 μg/ml (=1000 ng/ml) | |
| 3 | 1000 | ng/ml | 10 | 990 | 1 | ng/ml |
| 4 | 1 | ng/ml | 500 | 500 | 0.5 | ng/ml |
| 5 | 0.5 | ng/ml | 500 | 500 | 0.25 | ng/ml |
| 6 | 0.25 | ng/ml | 500 | 500 | 0.125 | ng/ml |
| 7 | 0.125 | ng/ml | 500 | 500 | 0.625 | ng/ml |
| 8 | | | | | | |

Dilution of Standard Polyamines: Spermine
and Spermidine for Calibration Curve
Spermidine: Sigma 4139-1G
Conc: 925 mg/ml

| | | | Volume | Solvent | | |
|---|---|---|---|---|---|---|
| | Stock | | (μl) | (μl) | Final | |
| 1 | 925 | mg/ml | 10.81 | 989.19 | 10 μg/ml (=10,000 ng/ml) | |
| 2 | 10 | μg/ml | 100 | 900 | 1 μg/ml (=1000 ng/ml) | |
| 3 | 1000 | ng/ml | 100 | 900 | 100 | ng/ml |
| 4 | 100 | ng/ml | 500 | 500 | 50 | ng/ml |
| 5 | 50 | ng/ml | 500 | 500 | 25 | ng/ml |
| 6 | 25 | ng/ml | 500 | 500 | 12.5 | ng/ml |
| 7 | 12.5 | ng/ml | 500 | 500 | 6.25 | ng/ml |
| 8 | | | | | | |

### Results

| | PUT | SPD | SPM | [PUT] | [SPD] | [SPM] |
|---|---|---|---|---|---|---|
| ID | (amount) | (amount) | (amount) | (concentration) | (concentration) | (concentration) |
| D-10-24 | 63429 | 2369255 | 2363841 | 0.030874 | 2.907779 | 7.638914 |
| D-50-24 | | 542711 | 1315606 | 0 | 0.666068 | 4.251471 |
| D-100-24 | | 42842 | 98016 | 0 | 0.05258 | 0.316745 |
| 1-10-24 | 14507 | 1202403 | 149654 | 0.007061 | 0.248409 | 0.483617 |
| 1-50-24 | | 163914 | 168544 | 0 | 0.201171 | 0.544661 |
| 1-100-24 | | 377626 | 500166 | | 0.463459 | 1.616321 |
| 3-10-24 | 56963 | 651020 | 481606 | 0.027726 | 0.798995 | 1.556343 |
| 3-50-24 | 379546 | 3615812 | 2470785 | 0.184742 | 4.437675 | 7.984511 |
| 3-100-24 | 27933 | 323941 | 212917 | 0.013596 | 0.397572 | 0.688056 |
| CNTRL 24 | 74845 | 661175 | 396101 | 0.03643 | 0.811458 | 1.280027 |
| D-10-48 | | 254356 | 373610 | 0 | 0.31217 | 1.207346 |
| D-50-48 | | 44007 | 482723 | 0 | 0.05401 | 1.559952 |
| D-100-48 | | 220260 | 7071485 | 0 | 0.270324 | 22.85199 |
| 1-10-48 | 43116 | 1887299 | 2659801 | 0.020986 | 2.316276 | 8.595329 |
| 1-50-48 | 23355 | 1774662 | 4106905 | 0.011368 | 2.178037 | 13.27174 |
| 1-100-48 | | 332304 | 1568383 | 0 | 0.407836 | 5.068337 |
| 3-10-48 | 124533 | 3898235 | 4032041 | 0.060616 | 4.784292 | 13.02982 |
| 3-50-48 | 34544 | 848798 | 693740 | 0.016814 | 1.041727 | 2.241868 |
| 3-100-48 | 7198 | 231781 | 236426 | 0.003504 | 0.284464 | 0.764027 |
| CNTRL 48 | 711717 | 14919476 | 12022888 | 0.346424 | 18.31063 | 38.85278 |
| STAND | 3389876 | 1344418 | 510588 | 1.65 | 1.65 | 1.65 |
| 1.65 | | | | | | |

ID KEY:

| | | |
|---|---|---|
| Position 1 | Position 2 | Position 3 |
| D = DFMO | 10 = 10 uM treatment | 24 = 24 hr treatment |
| 1 = DFMO Analog 1 | 50 = 50 uM treatment | 48 = 48 hour treatment |
| 3 = DFMO Analog 2 | 100 = 100 uM treatment | |

### Average Results—24 Hour Treatment

| 24 hr avg | put | spd | spm |
|---|---|---|---|
| DFMO | 0.010291217 | 1.208808867 | 4.069043 |
| DFMO Analog 1 | 0.002353729 | 0.304346304 | 0.881533 |
| DFMO Analog 2 | 0.075354703 | 1.878080441 | 3.409636 |
| Control | 0.036430315 | 0.811458006 | 1.280027 |

### Average Results—48 Hour Treatment

| 48 hr avg | put | spd | spm |
|---|---|---|---|
| DFMO | 0 | 0.212168128 | 8.539762 |
| DFMO Analog 1 | 0.010784775 | 1.634049641 | 8.97847 |
| DFMO Analog 2 | 0.026977757 | 2.036827609 | 5.345237 |
| Control | 0.346423601 | 18.31062616 | 38.85278 |

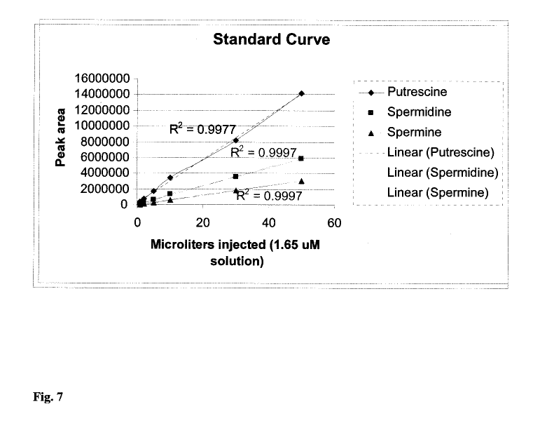

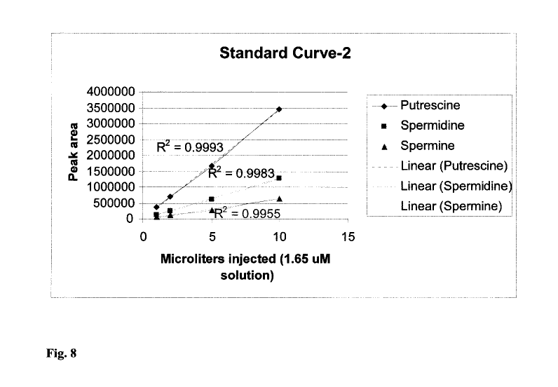

The results of the standard curve dilutions are shown in FIGS. 7 and 8.

### REFERENCES

Antony, T., et al. (2003) Cellular polyamines promote the aggregation of alpha-synuclein. J Biol Chem 278, 3235-3240.
Braak, H., et al. (2003) Staging of brain pathology related to sporadic Parkinson's disease. Neurobiol Aging 24, 197-211.
Braak H, G. E., Rüb U, Bratzke H, Del Tredici K. (2004), Stages in the development of Parkinson's disease-related pathology. Cell Tissue Res., 318(1): p. 121-134.
Casero, R. A., et al. (1989) Differential Induction of Spermidine/Spermine N1-Acetyltransferase in Human Lung Cancer Cells by the Bis(ethyl)polyamine Analogues. Cancer Res. 49, 3829-3833.
Chen, G., et al. (2002) Discordant protein and mRNA expression in lung adenocarcinomas. Mol Cell Proteomics 1, 304-313.
Conway, K. A., Harper, J. D., & Lansbury, P. T. (1998) Accelerated in vitro fibril formation by a mutant alpha-synuclein linked to early-onset Parkinson disease. Nat Med 4, 1318-1320.
Cooper, A. A., et al. (2006) α-Synuclein Blocks ER-Golgi Traffic and Rab1 Rescues Neuron Loss in Parkinson's Models. Science 313, 324-328.
Coppola, G. & Geschwind, D. H. (2006) Technology Insight: querying the genome with microarrays—progress and hope for neurological disease. Nature clinical practice 2, 147-158.
Dauer, W. & Przedborski, S. (2003) Parkinson's disease: mechanisms and models. Neuron 39, 889-909.
Dorsal Motor Nucleus of the Vagus (CNX). 2008 [cited; Available from: http://www.neuroanatomy.wisc.edu/virtualbrain/BrainStem/08CNX.html.
Fahn, S., Elton, R., & Committee., M. o. t. U. D. (1987), Recent Developments in Parkinson's Disease eds. Fahn, S., Marsden, C., Calne, D., & Goldstein, M. (Macmillan Healthcare Information), pp. 153-163, 293-304.
Fleming, S., et al. (2004) Early and Progressive Sensorimotor Anomalies in Mice Overexpressing Wild-Type Human alpha-Synuclein. J. Neurosci. 24, 9434-9440.
Forno, L., Neuropathology of Parkinson's disease. J. Neuropathol. Exp. Neurol., 1996. 55: p. 259-272.
Gerlach, M. and P. Riederer, Animal models of Parkinson's disease: an emperical comparison with the phenomenology of the disease in man. J. Neural Transm., 1996. 103: p. 987-1041.
Gemer, E. W. & Meyskens, F. L., Jr. (2004) Polyamines and cancer: old molecules, new understanding. Nature reviews 4, 781-792.
Gitler, A. D., et al. (2008) The Parkinson's disease protein α-synuclein disrupts cellular Rab homeostasis. Proc Natl Acad Sci 105, 145-150.
Gomes-Trolin, C., Nygren, I., Aquilonius, S. M., & Askmark, H. (2002) Increased red blood cell polyamines in ALS and Parkinson's disease. Exp Neurol 177, 515-520.
Grabenauer, M., et al. (2008) Spermine Binding to Parkinson's Protein α-Synuclein and Its Disease-Related A30P and A53T Mutants. J Phys Chem B. 112, 11147-11154.
Hardy, J., Cai, F L, Cookson, M. R., Gwinn-Hardy, K., & Singleton, A. (2006) Genetics of Parkinson's disease and parkinsonism. Ann Neurol 60, 389-398.
Ichimura, S., Nenoi, M., Mita, K., Fukuchi, K., & Hamana, K. (2004) Accumulation of spermidine/spermine N1-acetyltransferase and alternatively spliced mRNAs as a delayed response of HeLa S3 cells following X-ray irradiation. Int J Radiat Biol. 80, 369-375.
Ignatenko, N. A. et al. (2006) Pharmacogenomics of the Polyamin Analog 3,8,13,18-tetraaza-10,11-[(E)-1,2-cyclopropyl]eicosane Tetrahyrdochloride, CGC-11092, in the Colon Adenocarcinoma Cell Line HCT1161. TCRT 2006. Vol. 5, No. 6, 543-626 (ISSN1533-0338).
Kerr, M. K., Martin, M., & Churchill, G. A. (2000) Analysis of variance for gene expression microarray data. J Comput Biol 7, 819-837.
Kiegeris A., P. S., Giasson B. I., Maguire J., Zhang H., McGeer E. G., McGeer P. L., alpha-Synuclein activates stress signaling protein kinases in THP-1 cells and microglia. Neurobiol. Aging 2006. 11(13).
Klivenyi, P., et al., Mice lacking alpha-synuclein are resistant to mitochondrial toxins. Neurobiology of Disease, 2006. 21(3): p. 541-548.
Kontopoulos, E., J. D. Parvin, and M. B. Feany, {alpha}-synuclein acts in the nucleus to inhibit histone acetylation and promote neurotoxicity. 2006. p. 3012-3023.
Lee, H.-J., et al., Impairment of microtubule-dependent trafficking by overexpression of &#x03B1;-synuclein. 2006. p. 3153-3162.
Levy, G., Louis, E. D., Mejia-Santana, H, Cote, L., Andrews, H., Harris, J., Waters, C., Ford, B., Frucht, S., Fahn, S., et al. (2004) Lack of familial aggregation of Parkinson disease and Alzheimer disease, Arch Neurol 61, 1033-1039.
Lew, M., Overview of Parkinson's Disease. Pharmacotherapy, 2007. 27(12): p. 155S-160S.
Lewandowski, N. M. & Small, S. A. (2005) Brain microarray: Finding needles in molecular haystacks. J Neurosci 25, 10341-10346.
Li, J. Z., et al. (2004) Systematic changes in gene expression in postmortem human brains associated with tissue pH and terminal medical conditions. Hum Mol Genet 13, 609-616.
Libby, P. R. & Porter, C. W. (1992) Inhibition of enzymes of polyamine back-conversion by pentamidine and berenil. Biochem Pharmacol 44, 830-832.
Lin, W., Celik, A., & Paczynski, R. P. (1999) Regional cerebral blood volume: a comparison of the dynamic imaging and the steady state methods. J Magn Reson Imaging 9, 44-52.
Litvan, I., et al. (2007) The etiopathogenesis of Parkinson disease and suggestions for future research. Part I. J Neuropathol Exp Neurol 66, 251-257.
Marder, K., Levy, G., Louis, E. D., Mejia-Santana, H., Cote, L., Andrews, H., Harris, J., Waters, C., Ford, B., Frucht, S., et al. (2003A) Familial aggregation of early-and late-onset Parkinson's disease. Ann Neurol 54, 507-513.
Marder, K., Levy, G., Louis, E. D., Mejia-Santana, 1-1., Cote, L., Andrews, H., Harris, J., Waters, C., Ford, B., Frucht, S., et al. (2003B) Accuracy of family history data on Parkinson's disease. Neurology 61, 18-23.
Martin, L. J., et al., Parkinson's Disease {alpha}-Synuclein Transgenic Mice develop Neuronal Mitochondrial Degeneration and Cell Death. 2006. p. 41-50.
Marton, L. J. & Pegg, A. E. (1995) Polyamines as targets for therapeutic intervention. Annu Rev Pharmacol Toxicol 35, 55-91.
Moreno, H, et al. (2007) Imaging the abeta-related neurotoxicity of Alzheimer disease. Arch Neurol 64, 1467-1477.
Mosharov, E. V., et al., {alpha}-Synuclein Overexpression Increases Cytosolic Catecholamine Concentration. 2006. p. 9304-9311.
Muhammad, A., et al. (2008) Retromer deficiency observed in Alzheimer's disease causes hippocampal dysfunction, neurodegeneration, and Aβ accumulation. Proc Natl Acad Sci 105, 7327-7332.
Mytilineou, C., et al., Inhibition of proteasome activity sensitizes dopamine neurons to protein alterations and oxidative stress. J Neural Transm, 2004. 111(10-10): p. 1237-51.
Nieto, M., et al., Increased sensitivity to MPTP in human {alpha}-synuclein A30P transgenic mice. Neurobiology of Aging, 2006. 27(6): p. 848-856.
Niiranen, K., et al. (2006) Mice with targeted disruption of spermidine/spermine N1-acetyltransferase gene maintain nearly normal tissue polyamine homeostasis but show signs of insulin resistance upon aging. J Cell Mol Med. 10, 933-945.
Outeiro, T. F. & Lindquist, S. (2003) Yeast cells provide insight into alpha-synuclein biology and pathobiology. Science 302, 1772-1775.
Pfeiffer, R. F. (2003) Gastrointestinal dysfunction in Parkinson's disease. Lancet Neurol 2, 107-116.
Pierce, A. & Small, S. A. (2004) Combining brain imaging with microarray: isolating molecules underlying the physiologic disorders of the brain. Neurochem Res 29, 1145-1152.
Polymeropoulos, M. H., et al. (1997) Mutation in the alpha-synuclein gene identified in families with Parkinson's disease. Science 276, 2045-2047.
Porter, C. W., Ganis, B., Libby, P. R., & Bergeron, R. J. (1991) Correlations between Polyamine Analogue-induced Increases in Spermidine/Spennine N1-Acetyltransferase Activity, Polyamine Pool Depletion, and Growth Inhibition in Human Melanoma Cell Lines. Cancer Res. 51, 3715-3720.
Przuntek, H., Müller, Th and Riederer, P., Diagnostic staging of Parkinson's disease: conceptual aspects. J Neural Transm, 2004. 111(2): p. 201-216.
Rockenstein, E., et al. (2002) Differential neuropathological alterations in transgenic mice expressing alpha-synuclein from the platelet-derived growth factor and Thy-1 promoters. J Neurosci Research 68, 568-578.
Rogaeva, E., et al. (2007) The neuronal sortilin-related receptor SORL1 is genetically associated with Alzheimer disease. Nat Genet. 39, 168-177.
Schobel, S. A., Lewandowski, N. M., et al. (2009) Differential Targeting of the CA1 Subfield of the Hippocampal Formation by Schizophrenia and Related Psychotic Disorders. Arch Gen Psychiatry 66, 938-946.
Simuni, T. & Sethi, K. (2008) Nonmotor manifestations of Parkinson's disease. Ann Neurol 64, S65-S80.
Small, S., et al. (2005) Model-guided microarray implicates the retromer complex in Alzheimer's disease. Ann Neurol 58, 909-919.
Small, S. A. (2003) Measuring correlates of brain metabolism with high-resolution MRI: a promising approach for diagnosing Alzheimer disease and mapping its course. Alzheimer Dis Assoc Disord 17, 154-161.
Small. S. A. (2008) Retromer Sorting: A Pathogenic Pathway in Late-Onset Alzheimer Disease. Arch Neurol 65, 323-328.
Spillantini, M. G., et al., alpha-Synuclein in filamentous inclusions of Lewy bodies from Parkinson's disease and dementia with Lewy bodies. 1998. p. 6469-6473.
Sung, J. Y., et al., {alpha}-Synuclein overexpression reduces gap junctional intercellular communication in dopaminergic neuroblastoma cells. Neuroscience Letters, 2007. 416(3): p. 289-293.
Thomas, T. & Thomas, T. J. (2001) Polyamines in cell growth and cell death: molecular mechanisms and therapeutic applications. Cell Mol Life Sci. 58, 244-258.
Tomitori, H., et al. (2001) Multiple polyamine transport systems on the vacuolar membrane in yeast. Biochem. J 353, 681-688.
Veinbergs, I., et al. (2001) Role of Apolipoprotein E Receptors in Regulating the Differential in vivo Neurotrophic Effects of Apolipoprotein E. Exp Neurol. 170, 15-26.
Wallace, H. M. & Fraser, A. V. (2004) Inhibitors of polyamine metabolism: Review article. Amino Acids 26, 353-365.
Wallace, H. M. and Kiiranen, K. (2007) Polyamine analogues—an update. Amino Acids 33, 261-265.
Winner B., R. E., Lie D. C., Aigner R., Mante M., Bogdahn U., Couillard-Depres S., Masliah E., Winkler J., Mutant alpha-synuclein exacerbates age-related decrease of neurogenesis. Neurobiol. Aging 2006. 10(1016).
Witjas, T., Kaphan, E, Azulay, J P., Non-motor fluctuations in Parkinson's disease. Rev Neurol (Paris)., 2007. 163(8-9): p. 846-850.
Yarlett, N. et al. (2007) Acitivites of DL-α-Difluoromethylarginine and Polyamine Analogues against Cryptosporidium parvum Infection in a T-cell Receptor Alpha-Deficient Mouse Model. Antimicrobial Agents and Chemotherapy 51, 1234-1239.
Yeger-Lotem, E., et al. (2009) Bridging high-throughput genetic and transcriptional data reveals cellular responses to alpha-synuclein toxicity. Nat Genet. 41, 316-323.
Zoli, M., Pedrazzi, P., & Agnati, L. F. (1996) Regional and cellular distribution of spermidine/spermine N1-acetyltransferase (SSAT) mRNA in the rat central nervous system. Neurosci Lett 207, 13-16.
U.S. Pat. No. 7,312,244, issued Dec. 24, 2007.
U.S. Pat. No. 7,279,502 issued Oct. 9, 2007.
U.S. Pat. No. 7,186,825 issued Mar. 6, 2007.
U.S. Pat. No. 6,982,351 issued Jan. 3, 2006.
U.S. Pat. No. 7,453,011 issued Nov. 18, 2008.
U.S. Pat. No. 6,794,545 issued Sep. 21, 2004.
U.S. Pat. No. 6,649,587 issued Nov. 18, 2003.
U.S. Pat. No. 7,235,695 issued Jun. 26, 2007.
U.S. Pat. No. 6,872,852 issued Mar. 29, 2005.
U.S. Patent Application Publication No. 2004/0006049 published Jan. 8, 2004.
U.S. Patent Application Publication No. 2003/0130356 published Jul. 10, 2003.
U.S. Patent Application Publication No. 2009/0275664 published Nov. 5, 2009
U.S. Patent Application Publication No. 2009/0134456 published May 28, 2009.
U.S. Patent Application Publication No. 2007/0232677 published Oct. 4, 2007.
U.S. Patent Application Publication No. 2005/0027016 published Feb. 3, 2005
U.S. Patent Application Publication No. 2002/0143068 published Oct. 3, 2002.
U.S. Patent Application Publication No. 2002/0045780 published Apr. 18, 2002.

1. A method for treating a subject afflicted with a disease involving α-synucleic aggregation which comprises administering to the subject a compound which reduces the amount of polyamines and/or inhibits polyamine synthesis in the subject in an amount effective to reduce α-synucleic aggregation so as to thereby treat the subject.

2. The method according to claim 1, wherein the disease is Parkinson's Disease.

3. The method according to claim 1, wherein the compound reduces the amount of polyamines in the subject by inhibiting ornithine decarboxylase.

4. The method according to claim 3, wherein the compound is α-difluoromethylornithine (DFMO) or an analog of α-difluoromethylornithine.

5. (canceled)

6. The method according to claim 1, wherein the compound reduces the amount of polyamines in the subject by inducing SSAT1 synthesis in the subject.

7. The method according to claim 6, wherein the compound is an analog of a polyamine.

8. The method according to claim 7, wherein the compound is N1,N11-diethynorspermine (DENSPM).

9. The method according to claim 6, wherein the administration is intrathecal administration.

10-16. (canceled)

17. A method for a subject afflicted with a disease involving α-synucleic aggregation which comprises administering to the subject a compound which inhibits α-synucleic aggregation in the subject in an amount effective to reduce α-synucleic aggregation so as to thereby treat the subject.

18-26. (canceled)

27. A method for reducing the amount of α-synucleic aggregation in a brain cell which comprises treating the brain cell with a compound that reduces the amount of polyamines and/or inhibits polyamine synthesis in the brain cell so as to thereby reduce the amount of α-synucleic aggregation in the brain cell.

28-53. (canceled)

54. A compound having the structure 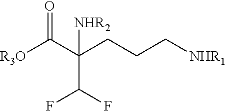
wherein R₁ is H, or
wherein R₄ is OH, OR₅, SR₅ or NHR₅
wherein R₅ is a substituted or unsubstituted alkyl, or a C₁-C₅ alkyl;
wherein R₂ is H,
wherein R₆ is OH, OR₇, SR₇, or NHR₇
wherein R₇ is a substituted or unsubstituted alkyl, or a C₃-C₅ alkyl;
wherein R₃ is H, a substituted or unsubstituted alkyl, or a C₁-C₅ alky;
or a salt or a pharmaceutically acceptable thereof.

55. The compound of claim 54, wherein
R₁ is H or 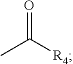
R₂ is H or 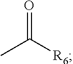
and R₃ is alkyl;
or a salt or a pharmaceutically acceptable salt thereof.

56-58. (canceled)

59. A compound having the structure: 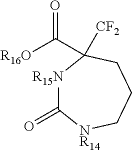
wherein
R₁₄ is H, substituted or unsubstituted alkyl, C₁-C₅ alkyl;
R₁₅ is H, substituted or unsubstituted alkyl, C₁-C₅ alkyl; and
R₁₆ is H, substituted or unsubstituted alkyl, C₁-C₅ alkyl;
or a salt or a pharmaceutically acceptable salt thereof.

60-61. (canceled)

62. A compound having the structure 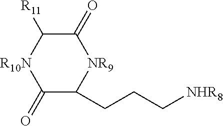
wherein
R₈ is H or 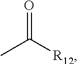
wherein R₁₂ is OH, OR₁₃, SR₁₃, or NHR₁₃
wherein R₁₃ is H, substituted or unsubstituted alkyl or C₁-C₅ alkyl;
R₉ is H or substituted or unsubstituted alkyl or C₁-C₅ alkyl;
R₁₀ is H or substituted or unsubstituted alkyl or C₁-C₅ alkyl; and
R₁₁ is H or substituted or unsubstituted alkyl;
or a salt or a pharmaceutically acceptable salt thereof.

63-64. (canceled)
